(19) United States
(12) Patent Application Publication
(10) Pub. No.: US 2003/0092616 A1
(43) Pub. Date: May 15, 2003

(54) STAT6 activation gene

(76) Inventors: Akio Matsuda, Shizuoka (JP); Goichi Honda, Shizuoka (JP); Shuji Muramatsu, Shizuoka (JP); Kenya Ishizawa, Shizuoka (JP)

Correspondence Address: BIRCH STEWART KOLASCH & BIRCH, PO BOX 747, FALLS CHURCH, VA, 22040-0747, US

(21) Appl. No.: 10/153,668

(22) Filed: May 24, 2002

Related U.S. Application Data
(60) Provisional application No. 60/293,172, filed on May 25, 2001.
(60) Provisional application No. 60/316,031, filed on Aug. 31, 2001.
(60) Provisional application No. 60/328,403, filed on Oct. 12, 2001.

(30) Foreign Application Priority Data
May 25, 2001 (JP) 157043/2001
Aug. 30, 2001 (JP) 260681/2001
Oct. 10, 2001 (JP) 313175/2001

(51) Int. Cl. A61K 38/17; C12P 21/02; C12N 5/06; C07H 21/04; C07K 14/435

(52) U.S. Cl. 514/12; 530/350; 435/69.1; 435/320.1; 435/325; 536/23.1

(57) ABSTRACT

Proteins having activity that promotes STAT6 activation, which are used for diagnosing, treating or preventing diseases associated with the excessive activation or inhibition of STAT6 are provided. Using a STAT6 response reporter plasmid, cDNA encoding a protein capable of promoting STAT6 activation was cloned from the cDNA library constructed from human lung fibroblasts, and the DNA sequence and the deduced amino acid sequence are determined. The protein, the DNA encoding the protein, a recombinant vector containing the DNA, and a transformant containing the recombinant vector are useful for screening a substance inhibiting or promoting STAT6 activation.

### TECHNICAL FIELD

The present invention relates to a protein capable of promoting STAT6 activation, a DNA sequence encoding the protein, a method for obtaining the DNA, a recombinant vector containing the DNA, a transformant containing the recombinant vector, and an antibody which reacts with the protein. The present invention also relates to use of the protein, DNA molecule or antibody of the invention in the diagnosis, treatment or prevention of diseases associated with the excessive activation or inhibition of STAT6.

The present invention also relates to a method for screening a substance capable of inhibiting or promoting STAT6 activation by using the protein, DNA, recombinant vector and transformant.

### BACKGROUND ART

Mosmann et al. advocated that helper T cells (the term will be abbreviated as “Th” hereinafter) which play an important role in immune response, should be classified into two different subsets (J. Immunol. (1986) 136:2348-2357). They classified these cells into two types of cell, Th1 and Th2 based on their cytokine-production pattern. Th1 cell produces interleukin2 (IL-2), interferon γ (IFN-γ), tumor necrosis factor β (TNF-β) etc., referred as Th1 type cytokines, and activates cell-mediated immunity, for example, in viral infection. On the other hand, Th2 cell produces interleukin4 (IL-4), interleukin5 (IL-5), interleukin10 (IL-10), interleukin13 (IL-13) etc., referred as Th2 type cytokines, and is involved in humoral immunity including infection of intracellular cytozoic microorganisms such as parasites and production of an antibody against exposure to an antigen/allergen. Thus, the idea of classifying various immune responses in a body depending on Th cell types to comprehend disease immune responses in view of the balance between Th1 and Th2 cells, has emerged, and a concept of Th1/Th2 diseases has also been suggested.

Since Th2 produces a number of cytokines involved in allergic reaction, hyperactive Th2 is considered to cause allergic disease such as asthma or the like.

IL-4 is an immunomodulatory cytokine which is secreted due to activation of T lymphocytes, basocytes, and mast cells. IL-4 induces proliferation of B cells and production of IgE and IgG1 as well as activation and proliferation of mast cells. It also induces gene expression of VCAM-1 which functions when a basocyte adheres to a vascular endothelical cell and infiltrates into tissues. Furthermore, IL-4 has been shown to play an important role in differentiation into a Th2 cell and proliferation and differentiation of a hemopoietic progenitor cell.

IL-13 is a cytokine secreted due to activation of T lymphocytes, mast cells, basocytes, NK cells, and dendritic cells. It has approximately 30% sequence identity to IL-4 and shows IL-4-like activity against monocyte/macrophage, B cell. However, IL-13 does not act on T cells.

Binding of IL-4 and IL-13 with their receptors on the cell surface activates intracellular tyrosine kinase, transmitting signals into the cell via tyrosine phosphorylation of some intracellular proteins. Recent developments in molecular biology have elucidated a signaling mechanism from the IL-4 receptor, and major intracellular transducer molecules have been identified. Among them, STAT6 has been found to be the most important molecule.

STAT6 is a member of a STAT (Signal transducer and Activator of Transcription) family. STAT is a transcription factor which functions depending on stimulations downstream of various cytokine receptors and growth factor receptors. In mammals, seven types, STATI, 2, 3, 4, 5a, 5b, and 6 have been identified so far. Binding of a ligand such as a cytokine with its receptor activates a receptor-associated tyrosine kinase referred as JAK family, and the activated JAK phosphorylates the tyrosine residues of the receptor itself, thereby causing activation of the STAT molecule. The activation of STAT6 molecule forms dimers and moves to the nucleus promptly, inducing gene expression.

JAK is activated via a IL-4 and a IL-13 receptor, and tyrosines on the receptors are phosphorylated. Subsequently, STAT6 binds to phosphorylated tyrosine residues of the receptors via SH2 domain, and STAT6 per se is tyrosine phosphorylated and forms homodimers, then moves to a nucleus. Known genes regulated by STAT6 include germline epsilon, CD23, MHC (Major Histocompatibility Complex) class II antigen, STAT6 gene, etc.

Recently, STAT6 defective mouse has been created and the physiological roles of STAT6 have been examined.

The fact that, in the STAT6 defective mouse, the major functions of IL-4 and IL-13 are all disturbed has demonstrated that STAT6 is a major molecule in signal transduction of IL-4 and IL-13. Further, the fact that Th2 reactions are disturbed in said mouse and that little production of Th2 type cytokine is confirmed demonstrated that STAT6 is also an essential molecule in Th2 cell differentiation.

Thus, STAT6 has been proved to be an important molecule in induction of allergic reaction.

In this context, the inhibition of function or activation of STAT6 may specifically inhibit the function of IL-4 and IL-13, repressing allergic disease, inflammatory or immunological diseases. Thus, the protein involved in STAT6 activation is a promising target for medicaments against diseases caused or characterized by allergic disease, autoimmunity or inflammation [see e.g., Proc. Natl. Acad. Sci. USA 95, 172-177 (1998), Science 282, 2258-2261 (1998), Science 282, 2261-2263 (1998), J. Exp. Med. 183, 109-117 (1996), J. Immunol. 160,4004-4009 (1998), J. Immunol. 160,1581-1588 (1998)].

Extracellular information is converted into a certain signal, which passes through the cell membrane and goes through the cytoplasm to the nucleus, where it regulates the expression of the target gene and causes cell responses. Therefore the elucidation of the mechanism of intracellular signal transduction from extracellular stimuli to STAT6 activation is of very important significance, because it provides very important means of developing new medicaments or therapies against autoimmune diseases and diseases exhibiting allergic disease, autoimmunity, or inflammatory symptoms.

It is considered, however, that the signal transduction pathway from a certain cell stimulis to STAT6 activation includes the existence of some other molecules which regulate and control the pathway in addition to JAK kinase and STAT molecule. Therefore it is desirable for more efficient drug discovery to identify the transmitters which play a key role in the pathway, and to focus research on the transmitters to establish a new drug-screening method. However, apart from JAK/STAT molecules, most of the mechanism of the signaling pathway via STAT6 remains unknown, and the identification of new signaling molecules and elucidation of the STAT6 activation mechanism are desired.

### DISCLOSURE OF TH INVENTION

The object of the present invention is to identify a new gene and protein capable of promoting STAT6 activation, and to provide a method of use of them in medicaments, diagnostics and therapy. That is, the present invention provides a new protein capable of promoting STAT6 activation, a DNA sequence encoding the protein, a recombinant vector containing the DNA, a transformant containing the recombinant vector, a process for producing the protein, an antibody directed against the protein or a peptide fragment thereof, and a process for producing the antibody.

The present invention also provides a method for screening a substance capable of inhibiting or promoting STAT6 activation, a kit for the screening, a substance capable of inhibiting or promoting STAT6 activation obtainable by the screening method or the screening kit, a process for producing the substance, a pharmaceutical composition containing a substance capable of inhibiting or promoting STAT6 activation, etc.

Recently, random analysis of cDNA molecules has been intensively carried out to analyze various genes, which are expressed in vivo. The cDNA fragments thus obtained have been entered for databases and published as ESTs (Expressed Sequence Tags, e.g., http//www.ncbi.nlm.nih.gov/dbEST). However, ESTs are merely sequence information, and it is difficult to predict their functions. ESTs are also arranged in UniGene (http//www.ncbi.nlm.nih.gov/UniGene), and about 80,000 human ETSs have been registered until now. However, most of these ESTs have their 5′ end nucleotide sequences deleted, and contain no translation initiation site. Therefore it is unlikely that such analysis will directly lead to gene functional analysis such as the analysis of protein functions on the assumption of the determination of mRNA coding regions and the understanding of gene expression control by the analysis of promoters.

On the other hand, one method to elucidate functions of gene products (i.e., proteins) is transient expression cloning method using animal cells [see e.g., “Idenshi Kougaku Handbook (Genetic Engineering Handbook)”, an extra issue of “Jikken Igaku (Experimental Medicine)”, YODOSHA CO., LTD.]. This method involves transfecting animal cells with a cDNA library constructed using an animal cell expression vector to directly express a functional protein, and identifying and cloning the cDNA based on the biological activity of the protein having an effect on the cells. This method requires no chemical information (amino acid sequences and molecular weights) regarding the target protein product as a prerequisite, and allows the identification of cDNA clones by detecting specific biological activity of the protein expressed in the cells or culture.

For the efficient expression cloning, there is a need to devise a method of preparing a cDNA library. Several methods have been widely used to construct cDNA libraries [e.g., the method of Gubbler-Hoffman: Gene 25 (1983); and the method of Okayama-Berg: Mol. Cell. Biol. 2 (1982)]. However, most of the cDNA molecules prepared by these methods have their 5′ end nucleotide sequences deleted, and thus these methods rarely produce full-length cDNA, a complete DNA copy of mRNA. This is because the reverse transcriptase used to prepare cDNA from mRNA does not necessarily have high efficiency in producing full-length cDNA. Therefore it is necessary to improve these prior art methods in order to efficiently carry out the above expression cloning.

In addition, in order to carry out the functional analysis of genes, it is essential to clone full-length cDNA sequences and express proteins from them. Therefore, it has been necessary to construct cDNA libraries containing enriched full-length cDNA for efficient expression cloning.

The present inventors have intensively studied to solve the above problems. As a result, the present inventors have succeeded in constructing a full-length cDNA library by using the oligo-capping method; establishing a gene function assay system by expression cloning using NIH3T3 cells; and isolating a new DNA (cDNA) encoding a protein having a function of promoting STAT6 activation by using the assay system. This new DNA molecule induced promotion of STAT6 activation by its expression in NIH3T3 cells. This result shows that this new DNA is a signal transduction molecule involved in promotion of STAT6 activation. Thus, the present invention has been completed.

That is, the present invention relates to:

(1) A purified and isolated protein selected from the group consisting of:

(a) a protein which consists of an amino acid sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482 and 484; and

(b) a protein that promotes STAT6 activation and consists of an amino acid sequence having at least one amino acid deletion, substitution or addition in an amino acid sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482 and 484.

(2) A purified and/or isolated protein that promotes STAT6 activation and comprises an amino acid sequence having at least 95% identity to any one of the proteins according to above item (1) over the entire length thereof,

(3) An isolated polynucleotide which consists of or comprises a nucleotide sequence encoding a protein selected from the group consisting of:

(a) a protein which comprises an amino acid sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482 and 484; and

(b) a protein that promotes STAT6 activation and consists of an amino acid sequence having at least one amino acid deletion, substitution or addition in an amino acid sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482 and 484;

(4) An isolated polynucleotide comprising a polynucleotide sequence selected from the group consisting of:

(a) a polynucleotide represented by any one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481 and 483; and a polynucleotide sequence complementary to said isolated polynucleotide;

(b) a polynucleotide sequence encoding a protein that promotes STAT6 activation and hybridizing with a polynucleotide having any one of the polynucleotide sequences of (a) under stringent conditions; and

(c) a polynucleotide sequence which encodes a protein that promotes STAT6 activation, and which consists of a polynucleotide sequence having at least one nucleotide deletion, substitution or addition in a polynucleotide sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481 and 483.

(5) An isolated polynucleotide comprising a nucleotide sequence which encodes a protein that promotes STAT6 activation and has at least 95% identity to any one of the polynucleotide sequences according to above item (3) over the entire length thereof;

(6) An isolated polynucleotide comprising a nucleotide sequence which encodes a protein that promotes STAT6 activation and has at least 95% identity to any one of the polynucleotide sequences according to above item (4) over the entire length thereof,

(7) A purified and/or isolated protein encoded by the polynucleotide according to any one of above items (3) to (6);

(8) A recombinant vector which comprises a polynucleotide according any one of above items (3) to (6).

(9) A transformed cell which comprises the recombinant vector according to above item (8).

(10) A membrane of the cell according to above item (9), when the protein according to above item (1) or (2) is a membrane protein.

(11) A process for producing a protein comprising,

(a) culturing a transformed cell comprising any one of the isolated polynucleotides according to any one of items (3) to (6), under conditions providing expression of the encoded protein; and

(b) recovering the protein from the culture product.

(12) A process for diagnosing a disease or a susceptibility to a disease in a subject related to expression or activity of the protein of item (1), (2) or (7) in a subject comprising:

(a) determining the presence or absence of a mutation in the nucleotide sequence encoding said protein in the genome of said subject; and/or

(b) analyzing the amount of expression of said protein in a sample derived from said subject, wherein a diagnosis of disease is made according to an increase or decrease in the amount of the protein expressed, wherein a diagnosis of disease is preferably made where the amount of protein expressed is 2-fold or higher than normal, or half or lower than normal.

(13) A method for screening a compound for activity as inhibitors or activators of STAT6, which comprises the steps of:

(a) providing a cell with a gene encoding a protein that promotes STAT6 activation, and a component that provides a detectable signal associated with activation of STAT6;

(b) culturing the transformed cell under conditions, which permit the expression of the gene in the transformed cell;

(c) contacting the transformed cell with one or more compounds; and

(d) measuring the detectable signal; and

(e) isolating or identifying as an activator compound and/or an inhibitor compound according to the detectable signal.

(14) A process for producing a pharmaceutical composition, which comprises the steps of:

(a) providing a cell with a gene encoding a protein that promotes STAT6 activation, and a component capable of providing a detectable signal;

(b) culturing the transformed cell under conditions, which permit the expression of the gene in the transformed cell;

(c) contacting the transformed cell with one or more candidate compounds;

(d) measuring the detectable signal; and

(e) isolating or identifying as an activator compound and/or an inhibitor compound according to the detectable signal; and

(f) optimizing the isolated or identified compound as a pharmaceutical composition.

(15) A kit for screening a compound for activity as an inhibitor or activator of STAT6, which comprises:

(a) a cell comprising a gene encoding a protein that promotes STAT6 activation, and a component that provides a detectable signal upon activation of STAT6; and

(b) reagents for measuring the detectable signal.

(16) A monoclonal or polyclonal antibody that reacts with the protein according to above item (1), (2) or (7).

(17) A process for producing a monoclonal or polyclonal antibody that reacts with the protein of above item (1), (2) or (7) which comprises administering the protein according to above item (1), (2) or (7) as an antigen or epitope-bearing fragments to a non-human animal.

(18) An antisense oligonucleotide complementary to the polynucleotide according to any one of above items (3) to (6), which prevents expression of protein that promotes STAT6 activation.

(19) A ribozyme which inhibits STAT6 activation by cleavage of RNA that encodes the protein of above item (1), (2) or (7), or by cleavage of RNA that encodes some protein of the pathway that leads to STAT6 activation.

(20) A method for treating a disease, which comprises administering to a subject an amount of a compound screened by the process according to above item (13), and/or a monoclonal or polyclonal antibody according to above item (16), and/or an antisense oligonucleotide according to above item (18), and/or a ribozyme according to above item (19) effective to treat a disease selected from the group consisting of allergic disease, inflammation, autoimmune diseases, diabetes, hyperlipidemia, infectious disease and cancers.

(21) A pharmaceutical composition produced according to item (14) as inhibiting or promoting STAT6 activation.

(22) A pharmaceutical composition according to item (21) for the treatment of allergic disease, inflammation, autoimmune diseases, cancers and viral infections.

(23) A method of treating allergic disease, inflammation, autoimmune diseases, cancers or viral infections, which comprising administering a pharmaceutical composition produced according to above item (14) to a patient suffering from a disease related to STAT6 activation.

(24) A pharmaceutical composition according to item (21) for the treatment of Th1 hyperactive diseases, for example, organ-specific autoimmune diseases such as multiple sclerosis and insulin-dependent diabetes mellitus, and rheumatism.

(25) A method of treating Th1 hyperactive diseases, for example, organ-specific autoimmune diseases such as multiple sclerosis and insulin-dependent diabetes mellitus, and rheumatism, which comprises administering a pharmaceutical composition produced according to above item (14) to a patient suffering a disease related to inhibition of STAT6 activation.

(26) A pharmaceutical composition which comprises a monoclonal or polyclonal antibody according to item (16)as an active ingredient.

(27) A pharmaceutical composition which comprises an antisense oligonucleotide according to item (18) as an active ingredient.

(28) The pharmaceutical composition according to item (26) or (27), wherein the target disease is selected from the group consisting of allergic disease, inflammation, autoimmune diseases, diabetes, hyperlipidemia, infections diseases and cancers.

(29) A computer-readable medium on which a sequence data set has been stored, said sequence data set comprising at least one nucleotide sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481 and 483, and/or at least one amino acid sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482 and 484.

(30) A method for calculating identity to other nucleotide sequences and/or amino acid sequences, which comprises comparing data on a medium according to above item (29) with data of said other nucleotide sequences and/or amino acid sequences.

(31) An insoluble substrate to which polynucleotide comprising all or part of the nucleotide sequences selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481 and 483 are fixed.

(32) An insoluble substrate to which polypeptides comprising all or a part of the amino acid sequences selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482 and 484 are fixed.

The contents of the specifications and/or drawings of Japanese Patent Applications Nos. 2001-157043, 2001-260681 and 2001-313175, and U.S. Provisional Applications Nos. 60/293,172, 60/316,031 and 60/328,403, which form the bases of priority of the instant application, are incorporated herein.

### BRIEF DESCRIPTION OF THE DRAWINGS

FIG. 1 is a graph showing STAT6 reporter activity inhibition by a protein kinase inhibitor, AG18, AG490, or staurosporin in the case where a plasmid comprising a nucleotide encoding a protein represented by SEQ ID NO: 3 which acts to promote STAT6 activation is used. In the figure, the vertical axis shows relative luciferase activity.

FIG. 2 is a graph showing STAT6 reporter activity inhibition by a protein kinase inhibitor, AG18, AG490, or staurosporin in the case where a plasmid comprising a nucleotide encoding a protein represented by SEQ ID NO: 17 which acts to promote STAT6 activation is used. In the figure, the vertical axis shows relative luciferase activity.

FIG. 3 is a graph showing STAT6 reporter activity inhibition by a protein kinase inhibitor, AG18, AG490, or staurosporin in the case where a plasmid comprising a nucleotide encoding a protein represented by SEQ ID NO: 19 which acts to promote STAT6 activation is used. In the figure, the vertical axis shows relative luciferase activity.

FIG. 4 is a graph showing STAT6 reporter activity inhibition by a protein kinase inhibitor, AG18, AG490, or staurosporin in the case where a plasmid comprising a nucleotide encoding a protein represented by SEQ ID NO: 218 which acts to promote STAT6 activation is used. In the figure, the vertical axis shows relative luciferase activity.

FIG. 5 is a graph showing STAT6 reporter activity inhibition by a protein kinase inhibitor, AG18, AG490, or staurosporin in the case where a plasmid comprising a nucleotide encoding a protein represented by SEQ ID NO: 432 which acts to promote STAT6 activation is used. In the figure, the vertical axis shows relative luciferase activity.

FIG. 6 is a graph showing STAT6 reporter activity inhibition by a protein kinase inhibitor, AG18, AG490, or staurosporin in the case where a plasmid comprising a nucleotide encoding a protein represented by SEQ ID NO: 472 which acts to promote STAT6 activation is used. In the figure, the vertical axis shows relative luciferase activity.

FIG. 7 is a graph showing STAT6 reporter activity inhibition by a protein kinase inhibitor, AG18, AG490, or staurosporin in the case where a plasmid comprising a nucleotide represented by SEQ ID NO: 64 is used. In the figure, the vertical axis shows relative luciferase activity.

### EXPLANATION OF THE SEQUENCE LISTING

SEQ ID NO: 485, SEQ ID NO: 486, SEQ ID NO: 487 and SEQ ID NO: 488 are primers.

### BEST MODE FOR CARRYING OUT THE INVENTION

At first, in order to further clarify the basic feature of the present invention, the present invention is explained by following how the present invention is completed. In order to obtain a new gene having a function of promoting STAT6 activation, the following experiments were carried out as shown in the examples. First, using the oligo-capping method, a full-length cDNA was produced from mRNA prepared from normal human lung fibroblasts (purchased from Sanko Junyaku Co., Ltd.), and a full-length cDNA library was constructed in which the cDNA was inserted into the vector pME18S-FL3 (GenBank Accession AB009864). Next, the cDNA library was introduced into E. coli cells, and plasmid preparation was carried out per clone. Then, a reporter plasmid containing a STAT6 response sequence upstream of DNA encoding luciferase (e.g., J. Biol. Chem. 275. 26500-26506 (2000), J. Exp. Med. 190, 1837-1848 (1999), J. Immunol. 150, 5408-5417 (1993), J. Immunol. 157, 2058-2065 (1996)) and the above full-length cDNA plasmid were cotransfected into NIH3T3 cells (Dainippon Pharmaceutical). After 48 hours of culture followed by slightly weak stimulation with mouse IL-4, luciferase activity was measured at a time of 6 hours thereafter, and the plasmid with significantly increased luciferase activity compared to that of a control experiment (vector pME18S-FL3 is introduced into a cell in place of a full-length cDNA) was selected (the selected plasmid showed a 3-fold or more increase in luciferase activity compared to that of the control experiment), and the entire nucleotide sequence of the cDNA cloned into the plasmid was determined. The protein encoded by the cDNA thus obtained shows that this protein is a signal transduction molecule involved in promotion of STAT6 activation.

The present invention is described in detail below.

In the present invention, the phrase “promote(s) STAT6 activation” means that direct or indirect activation of STAT6 (including induction of STAT6 activation) occurs when a gene is introduced into a suitable cell and the protein encoded by the gene is excessively expressed, without physiological stimuli; and/or that further direct or indirect promotion (including induction of promotion of STAT6 activation) of normal levels of STAT6 activation occurs, in the case where after the gene is introduced into a suitable cell and the protein encoded by the gene is excessively expressed, a physiological stimulus is introduced to the cell. Activation of STAT6 can be measured, for example, by an assay using an STAT6 dependant reporter gene. In the assay, activation can be detected by an increase in reporter activity compared to control cells (cells into which the reporter gene and a null vector were introduced). Increase in reporter activity is preferably by a factor of 1.5 or more, more preferably by a factor of 3 or more, and still more preferably by a factor of 6 or more.

Reporter activity can be measured by cloning a polynucleotide (e.g. cDNA) encoding the protein to be expressed into a suitable expression vector, co-transfecting the expression vector and a STAT6 dependant reporter plasmid into a suitable cell, and after culturing for a certain period, then measuring reporter activity. Or, after co-transfecting and culturing for a certain period, adding a stimulant, further culturing, then measuring reporter activity. Suitable expression vectors are well known to those skilled in the art, examples of which include pME18S-FL3, pcDNA3.1 (Invitrogen). The reporter gene can be one which enables a person skilled in the art to easily detect the expression thereof, and examples include a gene encoding luciferase, chloramphenicol acetyl transferase, or β-galactosidase. Use of a gene encoding luciferase is most preferable, and examples of an STAT6 dependent reporter plasmid include luciferase reporter plasmid N4×8-luc which has a STAT6 response sequence. Suitable cells include cells which exhibit an STAT6 activation response to stimulation by IL-4, IL-13 and the like. Examples include NIH3T3 cells. Cell culture and introduction of genes into cells (transfection) can be performed and optimized by a person skilled in the art by known techniques.

As a preferable method, NIH3T3 cells are inoculated on 10% FBS (Fetal Bovine Serum)-containing IMDM medium in a 96-well cell culture plate to a final cell density of 1×10⁴ cells/well, and cultured for 24 hours at 37° C., in the presence of 5% CO₂. Then, the luciferase reporter plasmid N4×8-luc which has a STAT6 response sequence, and the expression vector are cotransfected into the cells in a well using FuGENE 6 (Roche). After 48 hours of culture at 37° C., in the presence of 5% CO₂ mouse IL-4 (Immuno Biological Laboratories Co., Ltd.) is added to a final concentration of 0.5 ng/ml. After culturing for further 6 hours, promoting activity for STAT6 activation is then measured by measuring luciferase activity using a long term luciferase assay system, Picagene LT2.0 (Toyo Ink Mfg). For example, luciferase activity can be measured using PerkinElmer's Wallac ARVOTMST 1420 MULTILABEL COUNTER. The method for gene introduction by FuGENE6, and measurement of luciferase activity by Picagene LT2.0 can be performed respectively according to the attached protocols. In a method of gene introduction with a 96-well plate using FuGENE6, the amount of FuGENE6 per 1 well is suitably 0.3 to 0.5 μl, preferably 0.3 μl; the amount of N4×8-luc reporter plasmid is suitably 50 to 100 ng, preferably 100 ng; and the amount of expression vector is suitably 50 to 100 ng, preferably 100 ng. An ability (action) to promote STAT6 activation refers to an ability to increase the reporter activity (luciferase activity) relative to the control experiment (for cells into which the reporter gene and a null vector were introduced). Increase in reporter activity is preferably by a factor of 1.5 or more, more preferably by a factor of 3 or more, and still more preferably by a factor of 6 or more.

Related to the amino acid sequences of 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470. 472, 474, 476, 478, 480, 482 and 484 (hereinafter, sometimes referred to as amino acid sequences represented by SEQ ID NO: 1, etc), the present invention provides for a protein that:

(a) comprises the above amino acid sequences;

(b) is a polypeptide having one of the above amino acid sequences;

(c) promotes STAT6 activation and consists of an amino acid sequence having at least one amino acid deletion, substitution or addition in the above amino acid sequences:

(d) promotes STAT6 activation and comprises an amino acid sequence, which has at least 95% identity, preferably at least 97-99% identity, to the above amino acid sequences over the entire length thereof:

“Identity” as known in the art, is a relationship between two or more protein sequence or two or more polynucleotide sequences, as determined by comparing the sequences. In the art, “identity” also means the degree of sequence relatedness between protein or polynucleotide sequences, as determined by the match between protein or polynucleotide sequences, as the case may be, as determined by the match between strings of such sequences. “Identity” and “similarity” can be readily calculated by known methods. Preferred methods to determine identity are designed to give the largest match between the sequences tested. Methods to determine identity and similarity are codified in publicly available computer programs. “Identity” can be determined by using, for example, the BLAST program (for example, Altschul S F, Gish W, Miller W, Myers E W, Lipman D J., J. Mol. Biol., 215:p403-410(1990), Altschul S F, Madden T L, Schaffer A A, Zhang Z, Miller W, Lipman D J,. Nucleic Acids Res. 25:p3389-3402 81997)), however methods of determining identity are not limited to this. Where software such as BLAST is used, it is preferable to use default values.

The main initial conditions generally used in a BLAST search are as follows, but are not limited to these. An amino acid substitution matrix is a matrix numerically representing the degree of analogy of each pairing of each of the 20 types of amino acid, and normally the default matrix, BLOSUM62, is used. The theory of this amino acids substitution matrix is shown in Altschul S. F., J. Mol. Biol. 219: 555-565 (1991), and its applicability to DNA sequence comparison is shown in States D. J., Gish W., Altschul S. F., Methods, 3: 66-70 (1991). In this case, optimal gap cost is determined empirically and in the case of BLOSUM62, preferably parameters, Existence 11, Extension 1 are used.

The expected value (EXPECT) is the threshold value concerning statistical significance for a match with a database sequence, and the default value is 10.

As one example, a protein having, for example, 95% or more sequence identity to the amino acid sequence of SEQ ID NO: 1 may have an amino acid sequence that includes up to 5 amino acid changes per 100 amino acids of the amino acid sequence of SEQ ID NO: 1. In other words, a protein having 95% or more amino acid sequence identity to a subject amino acid sequence, may have amino acids up to 5% of the total number of amino acids within the subject sequence, deleted or substituted by other amino acids, or amino acids up to 5% of the total number of amino acids within the subject sequence may be inserted within the subject sequence. These changes within the subject sequence, may exist at the amino terminus or the carboxy terminus of the subject sequence, or may exist at any position between these termini, or may form one or more groups of changes.

The Examples described below demonstrate that the protein consisting of an amino acid sequence of the above SEQ ID NOS: 1, etc., is capable of promoting STAT6 activation.

Related to the polynucleotide sequences of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459; 461, 463, 465, 467, 469, 471, 473, 475, 477, 479, 481 and 483 (hereinafter, sometimes referred to SEQ ID NO: 2, etc), the present invention further provides an isolated polynucleotide that is:

(a) a polynucleotide of any of the above sequences;

(b) a polynucleotide comprising a polynucleotide sequence, which has at least 95% identity, preferably 97-99% identity, to any of the above sequences, and which encodes a protein which acts to promote STAT6 activation;

(c) a polynucleotide which has a nucleotide sequence that encodes a protein, wherein the protein has an amino acid sequence having at least 95% identity, preferably at least 97-99% identity, to the amino acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482 or 484 and acts to promote STAT6 activation.

Polynucleotides which are identical or sufficiently identical to a nucleotide sequence contained in the above nucleotide sequence may be used as hybridization probes to isolate full-length cDNA or genomic clones encoding proteins of the present invention or cDNA and genomic clones of other genes that have a high sequence similarity to the above sequences, or as primers for a nucleic acid amplification reactions. Typically, these nucleotide sequences are 70% identical, preferably 80% identical, more preferably 90% identical, most preferably 95% identical to the above sequences. The probes or primers will generally comprises at least 15 nucleotides, preferably 30 nucleotides and may have 50 nucleotides. Particularly preferred probes will have between 30 and 50 nucleotides. Particularly preferred primers have between 20 and 25 nucleotides.

The polynucleotide of the present invention may be either in the form of a DNA such as cDNA , a genomic DNA obtained by cloning or synthetically produced, or may be in the form of RNA such as mRNA. The polynucleotide may be single-stranded or double-stranded. The double-stranded polynucleotides may be double-stranded DNA, double-stranded RNA or DNA:RNA hybrid. The single-stranded polynucleotide may be sense strand also known as coding strand or antisense strand also known as non-coding strand.

Those skilled in the art can prepare a protein having the same activity that promotes STAT6 activation as the protein having an amino acid sequence represented by SEQ ID NO: 1, etc by means of appropriate substitution of an amino acid in the protein using known methods. One such method involves using conventional mutagenesis procedures for the DNA encoding the protein. Another method is, for example, site-directed mutagenesis (e.g., Mutan-Super Express Km Kit from Takara Shuzo Co., Ltd.). Mutations of amino acids in proteins may also occur in nature. Thus, the present invention also includes a mutated protein which is capable of promoting STAT6 activation and which has at least one amino acid deletion, substitution or addition compared to the protein having an amino acid sequence represented by SEQ ID NO: 1, etc. The number of mutations is preferably up to 10, more preferably up to 5, most preferably up to 3.

The substitutions of amino acids are preferably conservative substitutions, specific examples of which are substitutions within the following groups: (glycine, alanine), (valine, isoleucine, leucine), (aspartic acid, glutamic acid), (asparagine, glutamine), (serine, threonine), (lysine, arginine) and (phenylalanine, tyrosine).

Based on the nucleotide sequences (e.g., a polynucleotide of SEQ ID NO: 2, etc) encoding a protein consisting of an amino acid sequence of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482 or 484 or fragments thereof, those skilled in the art can routinely isolate a DNA with a high sequence similarity to these nucleotide sequences by using hybridization techniques and the like, and obtain proteins having the same activity that promotes STAT activation as the protein having of an amino acid sequence of SEQ ID NO: 1, etc. Thus, the present invention also includes a protein that promotes STAT6 activation and comprises an amino acid sequence having a high identity to the amino acid sequence of above SEQ ID NO: 1, etc. “High identity” refers to an amino acid sequence having an identity of at least 90%, preferably at least 97-99% over the entire length of an amino acid sequence represented by above SEQ ID NO: 1, etc.

The proteins of the present invention may be natural proteins derived from any human or animal cells or tissues, chemically synthesized proteins, or proteins obtained by genetic recombination techniques. The protein may or may not be subjected to post-translational modifications such as sugar chain addition or phosphorylation.

Examples of the protein of the present invention includes secretory proteins (growth factors, cytokines, hormones, etc.), protein modifying enzymes (protein phosphorylases, protein dephosphorylases, proteases, etc), intranuclear proteins (intranuclear receptors, transcription factors) and membrane proteins. Membrane proteins include receptors, cellular adhesion molecules, ion channels, transporters, etc. Where the protein is a membrane protein, a compound selected by the below-described screening is more useful as a medical compound research tool since it is expected to easily migrate into a cell.

The present invention also includes a polynucleotide encoding the above protein of the present invention. Examples of nucleotide sequences encoding a protein consisting of an amino acid sequence of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482 and 484 include nucleotide sequences of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481 and 483. The DNA includes cDNA, genomic DNA, and chemically synthesized DNA. In accordance with the degeneracy of the genetic code, at least one nucleotide in the nucleotide sequence encoding a protein consisting of an amino acid sequence of SEQ ID NOS: 1, 3, 5, 7, 9, 11, 13, 15, 17, 19 21, 23, 25, 27, 29, 31, 33, 35, 37, 39, 41, 43, 45, 47, 49, 51, 53, 55, 57, 59, 61, 63, 65, 67, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460 462, 464, 466, 468, 470, 472, 474, 476, 478, 480, 482 and 484 can be substituted with other nucleotides without altering the amino acid sequence of the protein produced from the gene. Therefore, the DNA sequences of the present invention also include nucleotide sequences altered by substitution based on the degeneracy of the genetic code. Such DNA sequences can be synthesized using known methods.

The DNA of the present invention includes a DNA which encodes a protein capable of promoting STAT6 activation and hybridizes under stringent conditions with the DNA sequence of the above nucleotide sequence of SEQ ID NO: 2, etc. Stringent conditions are apparent to those skilled in the art, and can be easily attained in accordance with various laboratory manuals such as T. Maniatis et al., Molecular Cloning A Laboratory Manual, and Cold Spring Harbor Laboratory 1982, 1989.

That is, “stringent conditions” refer to overnight incubation at 37° C. in a hybridization solution containing 30% formamide, 5×SSC (0.75 M NaCl, 75mM trisodium citrate), 5×Denhardt's solution, 0.5% SDS, 100 μg/ml denatured, sheared salmon sperm DNA) followed by washing (three times) in 2×SSC, 0.1% SDS for 10 minutes at room temperature, then followed by washing (two times) in 0.2×SSC, 0.1% SDS for 10 minutes at 37° C.(low stringency). Preferred stringent conditions are overnight incubation at 42 ° C. in a hybridization solution containing 40% formamide, followed by washing (three times) in 2×SSC, 0.1% SDS for 10 minutes at room temperature, then followed by washing (two times) in 0.2×SSC, 0.1% SDS for 10 minutes at 42° C.(moderate stringency). More preferred stringent conditions are overnight incubation at 42° C. in a hybridization solution containing 50% formamide, followed by washing (three times) in 2×SSC, 0.1% SDS for 10 minutes at room temperature, followed by washing (two times) in 0.2×SSC, 0.1% SDS for 10 minutes at 50° C. (high stringency). The DNA sequence thus obtained must encode a protein capable of promoting STAT6 activation.

The present invention also includes a polynucleotide comprising a nucleotide sequence which encodes a protein capable of promoting STAT6 activation and has a high sequence similarity to the nucleotide sequence of the polynucleotide according to above item (3) or (4). Typically these nucleotide sequence are 95% identical, preferably 97% identical, most preferably at least 99% identical to the nucleotide sequence of the polynucleotide according to above item (3) or (4) over the entire length thereof.

The above nucleotide sequence of the present invention can be used to produce the above protein using recombinant DNA techniques. In general, the DNA and peptide of the present invention can be obtained by:

(A) cloning the DNA encoding the protein of the present invention;

(B) inserting the DNA encoding the entire coding region of the protein or a part thereof into an expression vector to construct a recombinant vector;

(C) transforming host cells with the recombinant vector thus constructed; and

(D) culturing the obtained cells to express the protein or its analogue, and then purifying it by column chromatography.

General procedures necessary to handle DNA and recombinant host cells (e.g., E. coli) in the above steps are well known to those skilled in the art, and can be easily carried out in accordance with various laboratory manuals such as T. Maniatis et al., supra. All the enzymes, reagents, etc., used in these procedures are commercially available, and unless otherwise stated, such commercially available products can be used according to the use conditions specified by the manufactures' instructions to attain completely its objects. The above steps (A) to (D) can be further illustrated in more details as follows.

Techniques for cloning the DNA encoding the protein of the present invention include, in addition to the methods described in the specification of the present application, PCR amplification using a synthetic DNA having a portion of the nucleotide sequence of the present invention (e.g., SEQ ID NO: 2, etc), as a primer, and selection of the DNA inserted into a suitable vector by hybridization with a labeled DNA fragment encoding a partial or full coding region of the protein of the present invention or a labeled synthetic DNA. Another technique involves direct amplification from total RNAs or mRNA fractions prepared from cells or tissues, using the reverse transcriptase polymerase chain reaction (RT-PCR method). As a DNA inserted into a suitable vector, for example, a commercially available library (e.g., from CLONTECH and STRATAGENE) can be used. Techniques for hybridization are normally used in the art, and can be easily carried out in accordance with various laboratory manuals such as T. Maniatis et al., supra. Depending on the intended purpose, the cloned DNA encoding the protein of the present invention can be used as such or if desired after digestion with a restriction enzyme or addition of a linker. The DNA thus obtained may have a nucleotide sequence of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481 or 483 or a polynucleotide of above items (3) to (6). The DNA sequence to be inserted into an expression vector in the above step (B) may be a full-length cDNA or a DNA fragment encoding the above full-length protein, or a DNA fragment constructed so that it expresses a part thereof.

Thus, the present invention also includes a recombinant vector, which comprises the above DNA sequence. The expression vector for the protein of the present invention can be produced, for example, by excising the desired DNA fragment from the DNA encoding the protein of the present invention, and ligating the DNA fragment downstream of a promoter in a suitable expression vector.

Expression vectors for use in the present invention may be any vectors derived from prokaryotes (e.g., E. coli), yeast, fungi, insect viruses and vertebrate viruses so long as such vectors are replicable. However, the vectors should be selected to be compatible with microorganisms or cells used as hosts. Suitable combinations of host cell—expression vector systems are selected depending on the desired expression product.

When bacteria are used as hosts, plasmid vectors compatible with these bacteria are generally used as replicable expression vectors for recombinant DNA molecules.

For example, the plasmids pBR322 and pBR327can be used to transform E. coli. Plasmid vectors normally contain an origin of replication, a promoter, and a marker gene conferring upon a recombinant DNA a phenotype useful for selecting the cells transformed with the recombinant DNA. Example of such promoters include a β-lactamase promoter, lactose promoter and tryptophan promoter. Examples of such marker genes include an ampicillin resistance gene, and a tetracycline resistance gene. Examples of suitable expression vectors include the plasmids pUC18 and pUC19 in addition to pBR322, pBR327.

In order to express the DNA of the present invention in yeast, for example, YEp24 can be used as a replicable vector. The plasmid YEp24 contains the URA3 gene, which can be employed as a marker gene. Examples of promoters in expression vectors for yeast cells include promoters derived from genes for 3-phosphoglycerate kinase, glyceraldehyde-3-phosphate dehydrogenase and alcohol dehydrogenase.

Examples of promoters and terminators for use in expression vectors to express the DNA of the present invention in fungal cells include promoters and terminators derived from genes for phosphoglycerate kinase (PGK), glyceraldehyde-3-phosphate dehydrogenase (GAPD) and actin. Examples of suitable expression vectors include the plasmids pPGACY2 and pBSFAHY83.

Examples of promoters for use in expression vectors to express the DNA of the present invention in insect cells include a polyhedrin promoter and P10 promoter.

Recombinant vectors used to express the DNA of the present invention in animal cells normally contain functional sequences to regulate genes, such as an origin of replication, a promoter to be placed upstream of the DNA of the present invention, a ribosome-binding site, a polyadenylation site and a transcription termination sequence. Such functional sequences, which can be used to express the DNA of the present invention in eukaryotic cells, can be obtained from viruses and viral substances. Examples of such functional sequences include an SRα promoter, SV40 promoter, LTR promoter, CMV (cytomegalovirus) promoter and HSV-TK promoter. Among them, a CMV promoter and SR α promoter can be preferably used. As promoters to be placed inherently upstream of the gene encoding the protein of the present invention, any promoters can be used so long as they are suitable for use in the above host-vector systems. Examples of origins of replication include foreign origins of replication, for example, those derived from viruses such as adenovirus, polyoma virus and SV40 virus. When vectors capable of integration into host chromosomes are used as expression vectors, origins of replication of the host chromosomes may be employed. Examples of suitable expression vectors include the plasmids pSV2-dhfr (ATCC 37146), pBPV-1(9-1) (ATCC 37111), pcDNA3.1 (INVITROGEN) and pME18S-FL3.

The present invention also includes a transformed cell, which comprises the above recombinant vector.

Microorganisms or cells transformed with the replicable recombinant vector of the present invention can be selected from remaining untransformed parent cells based on at least one phenotype conferred by the recombinant vector. Phenotypes can be conferred by inserting at least one marker gene into the recombinant vector. Marker genes naturally contained in replicable vectors can be employed. Examples of marker genes include drug resistance genes such as neomycin resistance genes, and genes encoding dihydrofolate reductase.

As hosts for use in the above step (C), any of prokaryotes (e.g., E. coli), microorganisms (e.g., yeast and fungi) as well as insect and animal cells can be used so long as such hosts are compatible with the expression vectors used. Examples of such microorganisms include Escherichia coli strains such as E. coli K12 strain 294 (ATCC 31446), E. coli X1776 (ATCC 31537), E. coli C600, E. coli JM109 and E. coli B strain; bacterial strains belonging to the genus Bacillus such as Bacillus subtilis; intestinal bacteria other than E. coli, such as Salmonella typhimurium or Serratia marcescens; and various strains belonging to the genus Pseudomonas. Examples of such yeast include Saccharomyces cerevisiae, Schizosaccharomyces pombe, and Pichia pastoris. Examples of such fungi include Aspergillus nidulans, and Acremonium chrysogenum (ATCC 11550).

As insect cells, for example, Spodoptera frugiperda (Sf cells), High Five™ cells derived from eggs of Trichoplusiani, etc., can be used when the virus is AcNPV. Examples of such animal cells include HEK 293 cells, COS-1 cells, COS-7 cells, Hela cells, and Chinese hamster ovary (CHO) cells. Among them, CHO cells and HEK 293 cells are preferred.

When cells are used as hosts, combinations of expression vectors and host cells to be used vary with experimental objects. According to such combinations, two types of expression (i.e. transient expression and constitutive expression) can be included.

“Transformation” of microorganisms and cells in the above step (C) refers to introducing DNA into microorganisms or cells by forcible methods or phagocytosis of cells and then transiently or constitutively expressing the trait of the DNA in a plasmid or an intra-chromosome integrated form. Those skilled in the art can carry out transformation by known methods [see e.g., “Idenshi Kougaku Handbook (Genetic Engineering Handbook)”, an extra issue of “Jikken Igaku (Experimental Medicine)”, YODOSHA CO., LTD.]. For example, in the case of animal cells, DNA can be introduced into cells by known methods such as DEAE-dextran method, calcium-phosphate-mediated transfection, electroporation, lipofection, etc. For stable expression of the protein of the present invention using animal cells, there is a method in which selection can be carried out by clonal selection of the animal cells containing the chromosomes into which the introduced expression vectors have been integrated. For example, transformants can be selected using the above selectable marker as an indication of successful transformation. In addition, the animal cells thus obtained vising the selectable marker can be subjected to repeated clonal selection to obtain stable animal cell strains highly capable of expressing the protein of the present invention. When a dihydrofolate reductase (DHFR) gene is used as a selectable marker, one can culture animal cells while gradually increasing the concentration of methotrexate (MTX) and select the resistant strains, thereby amplifying the DNA encoding the protein of the present invention together with the DHFR gene to obtain animal cell strains having higher levels of expression.

The above transformed cells can be cultured under conditions which permit the expression of the DNA encoding the protein of the present invention to produce and accumulate the protein of the present invention. In this manner, the protein of the present invention can be produced. Thus, the present invention also includes a process for producing a protein, which comprises culturing a transformed cell comprising the isolated polynucleotide according to above item (3) to (6) under conditions providing expression of the encoded protein and recovering the protein from the culture.

The above transformed cells can be cultured by methods known to those skilled in the art (see e.g., “Bio Manual Series 4”, YODOSHA CO., LTD.). For example, animal cells can be cultured by various known animal cell culture methods including attachment culture such as Petri dish culture, multitray type culture and module culture, attachment culture in which cells are attached to cell culture carriers (microcarriers), suspension culture in which productive cells themselves are suspended. Examples of media for use in the culture include media commonly used for animal cell culture, such as D-MEM and RPMI 1640.

In order to separate and purify the protein of the present invention from the above culture, suitable combinations of per se known separation and purification methods can be used. Examples such methods include methods based on solubility, such as salting-out and solvent precipitation; methods based on the difference in charges, such as ion-exchange chromatography; methods mainly based on the difference in molecular weights, such as dialysis, ultrafiltration, gel filtration and SDS-polyacrylamide gel electrophoresis; methods based on specific affinity, such as affinity chromatography; methods based on the difference in hydrophobicity, such as reverse phase high performance liquid chromatography; and methods based on the difference in isoelectric points, such as isoelectric focusing. For example, a protein of the present invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxyapatite chromatography and lectin chromatography. Most preferably, high performance liquid chromatography is employed for purification. Well known techniques for refolding proteins may be employed to regenerate active conformation when the polypeptide is denatured during intracellular synthesis, isolation or purification.

The protein of the present invention can also be produced as a fusion protein with another protein. These fusion proteins are also included within the present invention. For the expression of such fusion proteins, any vectors can be used so long as the DNA encoding the protein can be inserted into the vectors and the vectors can express the fusion protein. Examples of proteins to which a polypeptide of the present invention can be fused include glutathione S-transferase (GST) and a hexa-histidine sequence (6×His). The fusion protein of the protein of the present invention with another protein can be advantageously purified by affinity chromatography using a substance with an affinity for the fusion partner protein. For example, fusion proteins with GST can be purified by affinity chromatography using glutathione as a ligand.

The present invention also includes an inhibitory protein, i.e., a protein capable of inhibiting the activity of the protein of above item (7). Examples of such inhibitory proteins include antibodies, or other proteins that bind to active sites of a protein of the above item (7), thereby inhibiting the expression of their activity.

The present invention also relates to an antibody that reacts with the protein of the present invention or a fragment thereof, and to production of such an antibody. More preferably, the present invention relates to an antibody that reacts specifically with the above-mentioned protein of the present invention or a fragment thereof. Herein, “specifically” refers to there being little, or preferably no, crossreactivity. The antibody is not specifically limited so long as it can recognize the protein of the present invention. Examples of such antibodies include polyclonal antibodies, monoclonal antibodies and their fragments, single chain antibodies and humanized antibodies. Antibody fragments can be produced by known techniques. Examples of such antibody fragments include, but not limited to, F(ab′)₂ fragments, Fab′ fragments, Fab fragments and Fv fragments. The antibody that specifically binds the protein of the present invention can be produced using the protein of the present invention or a peptide thereof as an immunogen according to per se known process for producing antibodies or antisera. For example, a monoclonal or polyclonal antibody can be produced by administering the protein according to above item (1) or (2) as an antigen or epitope-bearing fragments to a non-human animal. Such methods are described, for example, in “Shin Idenshi Kougaku Handbook (New Genetic Engineering Handbook)”, the third edition, an extra issue of “Jikken Igaku (Experimental Medicine)”, YODOSHA CO., LTD.

In the case of polyclonal antibodies, for example, the protein of the present invention or a peptide thereof can be injected to animals such as rabbits to produce antibodies directed against the protein or peptide, and then their blood can be collected. The polyclonal antibodies can be purified from the blood, for example, by ammonium sulfate precipitation or ion-exchange chromatography, or by using the affinity column on which the protein has been immobilized.

In the case of monoclonal antibodies, for example, animals such as mice are immunized with the protein of the present invention, their spleen is removed and homogenized to obtain spleen cells, which are then fused with mouse myeloma cells by using a reagent such as polyethylene glycol. From the resulting hybrid cells (i.e. hybridoma cells), the clone producing the antibody directed against the protein of the present invention can be selected. Then, the resulting clonal hybridoma cells can be implanted intraperitoneally into mice, the ascitic fluid recovered from the mice. The resulting monoclonal antibody can be purified, for example, by ammonium sulfate precipitation or ion-exchange chromatography, or by using the affinity column on which the protein has been immobilized.

When the resulting antibody is used to administer to humans, it is preferable to use a humanized antibody or human antibody in order to reduce its immunogenicity. These humanized antibodies or human antibodies can be produced using transgenic mice or other mammals. For a general review of humanized antibodies, see, for example, Morrison, S. L. et al., Proc. Natl. Acad. Sci. USA, 81:6851-6855 (1984); Jones, P. T. et al., Nature 321:522-525 (1986); Hiroshi Noguchi, Igaku no Ayumi (J. Clin. Exp. Med.) 167:457-462 (1993); Takashi Matsumoto, Kagaku to Seibutsu (Chemistry and Biology) 36:448-456 (1998). Humanized chimeric antibodies can be produced by linking a V region of a mouse antibody to a C region of a human antibody. Humanized antibodies can be produced by substituting a sequence derived from a human antibody for a region other than a complementarity-determining region from a mouse monoclonal antibody. In addition, human antibodies can be directly produced in the same manner as the production of conventional monoclonal antibodies by immunizing the mice whose immune systems have been replaced with human immune systems. These antibodies can be used to isolate or to identify clones expressing the protein or to purify the protein of the present invention from a cell extract or transformed cells producing the protein of the present invention. These proteins can also be used to construct ELISA, RIA (radioimmunoassay) and western blotting systems. These assay systems can be used for diagnostic purposes for detecting an amount of the protein of the present invention present in a body sample in a tissue or a fluid in the blood of an animal, preferably human. For example, they can be used for diagnosis of a disease characterized by undesirable activation of STAT6 resulting from (expression) abnormality of the protein of the present invention, such as allergic disease, inflammation, autoimmune disease, diabetes, hyperlipidemia, infection (for example, HIV infection), cancer and the like. In order to provide a basis for diagnosis of a disease, a standard value must be established. However, this is a well-known technique to those skilled in the art. For example, a method of calculating the standard value comprises binding a body fluid or a cell extract of normal individual of a human or an animal to an antibody against the protein of the present invention under a suitable condition for the complex formation, detecting the amount of the antibody-protein complex by chemical or physical means and then calculating the standard value for the normal sample using a standard curve prepared from a standard solution containing a known amount of an antigen (the protein of the present invention). The presence of a disease can be confirmed by deviation from the standard value obtained by comparison of the standard value with the value obtained from a sample of an individual latently suffering from a disease associated with the protein of the present invention. These antibodies can also be used as reagents for studying functions of the protein of the present invention.

The antibodies of the present invention can be purified and then administered to patients characterized by undesirable activation of STAT6 resulting from (expression) abnormality of the protein of the present invention, such as allergic disease, inflammation, autoimmune disease, diabetes, hyperlipidemia, infection (such as HIV infection), cancer and the like. Thus in another aspect, the present invention is a pharmaceutical composition which comprises the above antibody as an active ingredient, and therapy using the antibody of the present invention. In such pharmaceutical compositions, the active ingredient may be combined with other therapeutically active ingredients or inactive ingredients (e.g., conventional pharmaceutically acceptable carriers or diluents such as immunogenic adjuvants) and physiologically non-toxic stabilizers and excipients. The resulting combinations can be sterilized by filtration, and formulated into vials after lyophilization or into various dosage forms in stabilized and preservable aqueous preparations. Administration to a patient can be intra-arterial administration, intravenous administration and subcutaneous administration, which are well known to those skilled in the art. The dosage range depends upon the weight and age of the patient, route of administration and the like. Suitable dosages can be determined by those skilled in the art. These antibodies exhibit therapeutic activity by inhibiting the promotion of STAT6 activation mediated by the protein of the present invention.

The DNA of the present invention can also be used to isolate, identify and clone other proteins involved in intracellular signal transduction processes. For example, the DNA sequence encoding the protein of the present invention can be used as a “bait” in yeast two-hybrid systems (see e.g., Nature 340:245-246 (1989)) to isolate and clone the sequence encoding a protein (“prey”) which can associate with the protein of the present invention. In a similar manner, it can be determined whether the protein of the present invention can associate with other cellular proteins (e.g., STAT6, JAK1). In another method, proteins which can associate with the protein of the present invention can be isolated from cell extracts by immunoprecipitation [see e.g., “Shin Idenshi Kougaku Handbook (New Genetic Engineering Handbook)”, an extra issue of “Jikken Igaku (Experimental Medicine)”, YODOSHA CO., LTD.] using antibodies directed against the protein of the present invention. In still another method, the protein of the present invention can be expressed as a fusion protein with another protein as described above, and immunoprecipitated with an antibody directed against the fusion protein in order to isolate a protein which can associate with the protein of the present invention.

The diagnostic assays offer a process for diagnosing or determining a susceptibility to the diseases through detection of mutation in the nucleotide sequence encoding STAT6 activation-promoting protein by the methods described. In addition, such diseases may be diagnosed by methods comprising determining from a sample derived from a subject an abnormally decreased or increased level of protein or mRNA. Decreased or increased expression can be measured at the RNA level using any of the methods well known in the art for the quantitation of polynucleotides, such as, for example, nucleic acid amplification, for instance PCR, RT-PCR, RNase protection method, Northern blotting and other hybridization methods. Assay techniques that can be used to determine levels of a protein in a sample derived from a host are well-known to those skilled in the art. Such assay methods include radioimmunoassays, competitive-binding assays, Western blot analysis and ELISA assays.

The DNA of the present invention can be used to detect abnormality in the DNA or mRNA encoding the protein of the present invention or a peptide fragment thereof. The invention relates to a method for diagnosing a disease, or susceptibility to a disease associated with the expression of the protein according to above item (1), (2) or (7) in a subject, which comprises determining mutations in the polynucleotide sequence encoding the protein. Thus, for example, the DNA of the present invention is useful for gene diagnosis regarding damage, mutations, and reduced, increased or over-expression of the DNA or mRNA. That is, the present invention includes a method for diagnosing a disease associated with the expression or activity of said protein in a subject, which comprises the steps of:

A process for diagnosing a disease or susceptibility to a disease in a subject related to expression or activity of the protein of above item (1), (2) or (7) in a subject comprising:

(a) determining the presence or absence of a mutation in the nucleotide sequence encoding said protein in the genome of said subject; and/or

(b) analyzing the amount of expression of said protein in a sample derived from said subject, wherein a diagnosis of disease is made when the amount of the protein expressed is 2-fold or higher than normal, or half or lower than normal.

When the nucleotide sequence encoding STAT6 activation-promoting protein contains a mutation according to the above step (a), the mutation may cause disease associated with the expression or activity of STAT6. When the amount of the expression of the protein of above item (1), (2) or (7) is different from the normal value according to the above step (b), the abnormal expression of the STAT6 activation-promoting new protein of the present invention may be responsible for diseases associated with the expression or activity of STAT6. Determination of the presence or absence of a mutation in the nucleotide sequence encoding STAT6 activation-promoting protein in the above step (a) may involve RT-PCR using a part of the nucleotide sequence encoding said protein as a primer, followed by conventional DNA sequencing to detect the presence or absence of the mutation. PCR-SSCP [Genomics 5:874-879 (1989); “Shin Idenshi Kougaku Handbook (New Genetic Engineering Handbook)”, an extra issue of “Jikken Igaku (Experimental Medicine)”, YODOSHA CO., LTD.] can also be used to determine the presence or absence of the mutation. Measurement of the amount of the expression of the protein in the above step (b) may involve, for example, using the antibody of above item (16).

The present invention also relates to a method for screening compounds for activity as inhibitors or promoters of STAT6 activation.

It should be noted that compounds that inhibit STAT6 activation, will, as a result of this action, have in vivo and in vitro activity as a STAT6 inhibiting agent. Also, compounds that promote STAT6 activation, will, as a result of this action, have in vivo and in vitro activity as a STAT6 activating agent. Consequently, the above screening method is for screening in respect of activity as an inhibiting agent or activating agent of STAT6, and the above compound is a compound having activity as an inhibiting agent or activating agent of STAT6.

The above screening method comprises the following steps:

(a) providing a cell with a gene encoding a protein that promotes STAT6 activation, and a component that provides a detectable signal upon activation of STAT6;

(b) culturing the transformed cell under conditions, which permit the expression of the gene in the transformed cell;

(c) contacting the transformed cell with one or more compounds; and

(d) measuring the detectable signal; and

(e) isolating or identifying as an activator compound and/or inhibitor compound according to the detectable signal.

A compound that increases the detectable signal 2-fold or higher than normal is preferably isolated or identified as an activator compound, and a compound that decreases the detectable signal 80% or less than normal is preferably isolated or identified as an inhibitor compound.

Examples of components capable of providing a detectable signal include reporter genes. Reporter genes are used instead of directly detecting the activation of transcription factors of interest. The transcriptional activity of a promoter of a gene is analyzed by linking the promoter to a reporter gene and measuring the activity of the product of the reporter gene (“Bio Manual Series 4” (1994), YODOSHA CO., LTD.).

Any peptide or protein can be used so long as those skilled in the art can measure the activity or amount of the expression product (including the amount of the produced mRNA) of the reporter genes. For example, enzymatic activity of chloramphenicol acetyltransferase, β-galactosidase, luciferase, etc., can be measured. Any reporter plasmids can be used to evaluate STAT6 activation so long as the reporter plasmids have an STAT6 recognition sequence inserted upstream of the reporter gene. For example, a sequence derived from the CD23 or germline C epsilon transcription initiation site can be used. Other examples include reporter plasmids described in J. Biol. Chem. 275, 26500-26506 (2000), J. Exp. Med. 190, 1837-1848 (1999), J. Immunol. 150, 5408-5417 (1993), J. Immunol. 157, 2058-2065 (1996).

Any host cells can be used so long as promotion of STAT6 activation can be detected in the host cells. Preferred host cells are mammalian cells such as NIH3T3 cells, HepG2 cells and the like. Transformation and culture of the cells can be carried out as described above.

In a specific embodiment, the method for screening a compound which inhibits or promotes STAT6 activation comprises culturing the transformed cell for a certain period of time, adding a certain amount of a test compound, measuring the reporter activity expressed by the cell after a certain period of time, and comparing the activity with that of a cell to which the test compound has not been added. On this occasion, proper stimulation, e.g. addition of IL-4, etc. may be optionally carried out at the same time. The reporter activity can be measured by methods known in the art (see e.g., “Bio Manual Series 4” (1994), YODOSHA CO., LTD.). Examples of test compounds include, but not limited to, low molecular weight compounds and peptides. Test compounds may be artificially synthesized compounds or naturally occurring compounds. Test compounds may be a single compound or mixtures. Examples of such detectable signals which may be measured include the amount of mRNA or proteins for genes whose expression is known to be induced accordingly by STAT6 activation (e.g., genes for IL-1 Receptor Antagonist, CD23, MHC Class II and STAT6) in addition to the above reporter genes. Activated STAT6 can also be quantified by a method for detecting bindings of DNA and protein such as gel mobility shift assay, etc. Alternatively, phosphorylation of STAT6 can be quantified with cell extracts.

The amount of mRNA can be measured, for example, by northern hybridization, RT-PCR, etc. The amount of proteins can be measured, for example, by using antibodies. The antibodies may be produced by known methods. Commercially available antibodies(from, e.g., Wako Pure Chemical Industries, Ltd.) can also be used.

It is also possible to produce a pharmaceutical composition according to the following steps (a) to (f):

(a) providing a cell with a gene encoding a protein that promotes STAT6 activation, and a component that provides a detectable signal upon activation of STAT6;

(b) culturing the transformed cell under conditions, which permit the expression of the gene in the transformed cell;

(c) contacting the transformed cell with one or more candidate compounds;

(d) measuring the detectable signal; and

(e) isolating or identifying as an activator compound and/or an inhibitor compound according to the detectable signal; and

(f) optimizing the isolated or identified compound as a pharmaceutical composition.

The protein of the present invention may also be used in a method for the structure-based design of an agonist, antagonist or inhibitor of the protein, by:

(a) determining in the first instance the three-dimensional structure of the protein;

(b) deducing the three-dimensional structure for the likely reactive or binding site(s) of an agonist, antagonist or inhibitor;

(c) synthesising candidate compounds that are predicted to bind to or react with the deduced binding or reactive site; and

(d) testing whether the candidate compounds are indeed agonists, antagonists or inhibitor.

The present invention also includes a compound obtainable by the above screening method. However, the screening method of the present invention is not limited to the above method. The present invention also includes a process for producing the pharmaceutical composition by the method of above item (14).

There is no special limitation to the above candidate compounds. Such compounds include low molecular weight compounds and peptides. They may be artificially synthesised compounds and naturally occurring compounds. As the compounds obtained by the above screening methods have a function as inhibiting or promoting STAT6 activation, they are useful as therapeutic or preventive pharmaceuticals for the treatment of diseases resulting from unfavorable activation or inactivation of STAT6. In order to isolate and purify the target compounds from the mixture, it is suitable to combine the known methods such as filtration, extraction, washings, drying, concentration, crystallization, various chromatography. When obtainment of a salt of the compounds is desired, a compound which is obtained in the form of a salt can be purified as it is. A compound which is obtained in the free form can be converted into a salt by isolating and purifying a salt obtained by dispersing or dissolving the compound into a suitable solvent and then adding a desired acid or base. Examples of a step to optimize the compounds or salts thereof obtained by the method of the present invention as a pharmaceutical composition, include methods of formulating according to ordinary processes such as the following. The above compounds or their pharmaceutically acceptable salts in an amount effective as an active ingredient, and pharmaceutically acceptable carriers can be mixed. Further, a form of formulation suitable for the selected mode of administration is selected. A composition suitable for oral administration includes a solid form such as tablet, granule, capsule, pill and powder, and solution form such as solution, syrup, elixir and dispersion. A form useful for parenteral administration includes sterile solution, dispersion, emulsion and suspension. The above carriers include, for example, sugars such as gelatin, lactose and glucose, starches such corn, wheat, rice and maize, fatty acids such as stearic acid, salts of fatty acids such as calcium stearate, magnesium stearate, talc, vegetable oil, alcohol such as stearyl alcohol and benzyl alcohol, gum, and polyalkylene glycol. Examples of such liquid carriers include generally water, saline, sugar solution of dextrose and the like, glycols such as ethylene glycol, propylene glycol and polyethylene glycol.

The present invention also includes a kit for screening compounds for activity as an inhibitor or promoter of STAT6 activation. The kit comprises reagents and the like necessary for screening compounds for inhibiting or promoting activity for STAT6 activation, including:

(a) a cell comprising a gene encoding a protein that promotes STAT6 activation, and a component that provides a detectable signal enabling detection of STAT6 activation after activation of STAT6; and

(b) reagents for measuring the detectable signal.

In another aspect, the present invention relates to a diagnostic kit which comprises:

(a) a polynucleotide of the present invention having a nucleotide sequence represented by any one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481 and 483;

(b) a polynucleotide having a nucleotide sequence complementary to that of (a);

(c) a protein of the present invention having an amino acid sequence represented by any one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482 and 484 or a fragment thereof; or

(d) an antibody to a protein of the present invention of (c).

A kit comprising at least any one of (a) to (d) is useful for diagnosing a disease or susceptibility to a disease such as allergic disease, inflammation, autoimmune diseases, diabetes, hyperlipidemia, infectious diseases (e.g., HIV infection) and cancers.

Because STAT6 is involved in a wide variety of pathological conditions such as allergic disease, inflammation, autoimmune diseases, diabetes, hyperlipidemia, cancers and viral infections, it is an attractive target for drug design and therapeutic intervention. Many experiments show that the inhibition of STAT6 activity may have significant physiological effects [see e.g., Nature 380, 627-630 (1996), Nature 380, 630-633 (1996), Immunity 4, 313-319 (1996), J. Immunol. 157, 3220-3222 (1996), Immunity 8, 255-264 (1998), J. Exp. Med. 187, 939-948 (1998), J. Exp. Med. 187, 1537-1542 (1998)] The finding of the new protein described herein capable of promoting STAT6 activation has provided a new method for inhibiting an abnormal STAT6 function. Thus, the present invention also relates to use of a compound which inhibits the function of the protein capable of promoting STAT6 activation described above, for inhibiting STAT6 activation. The compound obtained by the above screening method, which inhibits STAT6 activation, is useful as a medicament to treat or prevent diseases characterized by undesirable activation of STAT6, such as allergic disease, inflammation, autoimmune diseases, diabetes, hyperlipidemia, infectious diseases (e.g., HIV infection) and cancers.

On the other hand, since STAT6 activation promotes differentiation into Th2 cells, there is also a possibility of reducing symptoms of or treating Th1 hyperactive diseases, for example, organ-specific autoimmune diseases such as multiple sclerosis and insulin-dependent diabetes mellitus, and rheumatism. Thus, the compound obtained by the above screening method, which promotes STAT6 activation, is useful as a medicament to treat or prevent these diseases.

In addition, the gene encoding the protein of the present invention is useful for gene therapy to treat various diseases such as cancers, autoimmune diseases, diabetes, hyperlipidemia, allergy diseases and inflammatory response. “Gene therapy” refers to administering into the human body a gene or a cell into which a gene has been introduced. The protein of the present invention and the DNA encoding the protein can also be used for diagnostic purposes.

The compound obtained by the screening method of the present invention or a salt thereof can be formulated into the above pharmaceutical compositions (e.g., tablets, capsules, elixirs, microcapsules, sterile solutions and suspensions) according to conventional procedures. The formulations thus obtained are safe and of low toxicity, and can be administered, for example, to humans and mammals (e.g., rats, rabbits, sheep, pigs, cattle, cats, dogs and monkeys). Administration to patients can be carried out by methods known in the art, such as intra-arterial injection, intravenous injection and subcutaneous injection. The dosage may vary with the weight and age of the patient as well as a mode of administration, but those skilled in the art can appropriately select suitable dosages. When the compound can be encoded by DNA, the DNA can be inserted into a vector for gene therapy, and gene therapy can be carried out. The dosage and mode of administration may vary with the weight, age and symptoms of the patient, but those skilled in the art can appropriately select them. Thus, the present invention also relates to a pharmaceutical composition which comprises the above compound as an active ingredient.

In addition, the above compound is useful as a medicament to treat or prevent diseases characterized by undesirable activation of STAT6, such as allergic disease, inflammation, autoimmune diseases, diabetes, hyperlipidemia, viral diseases, infectious diseases and cancers. Thus, the present invention also relates to a pharmaceutical composition for allergic disease, inflammation, autoimmune diseases, diabetes, hyperlipidemia, viral diseases, cancers, etc., which comprises the above compound. Specifically, the pharmaceutical composition is useful as a therapeutic and prophylactic drug against, for example, rheumatoid arthritis, osteoarthritis, systemic lupus erythematosus, diabetes, sepsis, asthma, allergic rhinitis, ischemic heart diseases, inflammatory intestinal diseases, subarachnoid hemorrhage, viral hepatitis and AIDS.

The present invention also relates to the use of a pharmaceutical composition produced according to above item (14) for manufacturing a medicament against allergic disease, inflammation, autoimmune diseases, diabetes, hyperlipidemia, viral diseases, cancers, etc.

The present invention also includes an antisense oligonucleotide against a gene of any one of above items (3) to (6). An antisense oligonucleotide refers to an oligonucleotide complementary to the target gene sequence. The antisense oligonucleotide can inhibit the expression of the target gene by inhibiting RNA functions such as translation to proteins, transport to the cytoplasm and other activity necessary for overall biological functions. In this case, the antisense oligonucleotide may be RNA or DNA. The DNA sequence of the present invention can be used to produce an antisense oligonucleotide capable of hybridizing with the mRNA transcribed from the gene encoding the protein of the present invention. It is known that an antisense oligonucleotide generally has an inhibitory effect on the expression of the corresponding gene (see e.g., Saibou Kougaku Vol.13, No.4 (1994)). The oligonucleotide containing an antisense coding sequence against a gene encoding the protein of the present invention can be introduced into a cell by standard methods. The oligonucleotide effectively blocks the translation of mRNA of the gene encoding the protein of the present invention, thereby blocking its expression and inhibiting undesirable activity.

The oligonucleotide of the present invention may be a naturally occurring oligonucleotide or its modified form [see e.g., Murakami & Makino, Saibou Kougaku Vol.13, No.4, p.259-266 (1994); Akira Murakami, Tanpakushitsu Kakusan Kouso (PROTEIN, NUCLEIC ACID AND ENZYME) Vol.40, No.10, p.1364-1370 (1995), Tunenari Takeuchi et al., Jikken Igaku (Experimental Medicine) Vol.14, No.4 p85-95(1996)]. Thus, the oligonucleotide may have modified sugar moieties or inter-sugar moieties. Examples of such modified forms include phosphothioates and other sulfur-containing species used in the art. According to several preferred embodiments of the present invention, at least one phosphodiester bond in the oligonucleotide is substituted with the structure which can enhance the ability of the composition to permeate cellular regions where RNA with the activity to be regulated is located.

Such substitution preferably involves a phosphorothioate bond, a phosphoramidate bond, methylphosphonate bond, or a short-chain alkyl or cycloalkyl structure. The oligonucleotide may also contain at least some modified base forms. Thus, it may contain purine and pyrimidine derivatives other than naturally occurring purine and pyrimidine. Similarly, the furanosyl moieties of the nucleotide subunits can be modified so long as the essential purpose of the present invention is attained. Examples of such modifications include 2′-O-alkyl and 2′-halogen substituted nucleotides. Examples of modifications in sugar moieties at their 2-position include OH, SH, SCH₃, OCH₃, OCN or O(CH₂)ₙCH₃, wherein n is 1 to about 10, and other substituents having similar properties. All the analogues are included in the scope of the present invention so long as they can hybridize with the mRNA of the gene of the present invention to inhibit functions of the mRNA.

The oligonucleotide of the present invention contains about 3 to about 50 nucleotides, preferably about 8 to about 25 nucleotides, more preferably about 12 to about 20 nucleotides. The oligonucleotide of the present invention can be produced by the well-known solid phase synthesis technique. Devices for such synthesis are commercially available from some manufactures including Applied Biosystems. Other oligonucleotides such as phosphothioates can also be produced by methods known in the art.

The oligonucleotide of the present invention is designed to hybridize with the mRNA transcribed from the gene of the present invention. Those skilled in the art can easily design an antisense oligonucleotides based on a given gene sequence (For example, Murakami and Makino: Saibou Kougaku Vol. 13 No.4 p259-266 (1994), Akira Murakami: Tanpakushitsu Kakusan Kouso (PROTEIN, NUCLEIC ACID AND ENZYME) Vol. 40 No.10 p1364-1370 (1995), Tunenari Takeuchi et al., Jikken Igaku (Experimental Medicine) Vol. 14 No. 4 p85-95 (1996)). Recent study suggests that antisense oligonucleotides which are designed in a region containing 5′ region of mRNA, preferably, the translation initiation site, are most effective for the inhibition of the expression of a gene. The length of the antisense oligonucleotides is preferably 15 to 30 nucleotides and more preferably 20 to 25 nucleotides. It is important to confirm no interaction with other mRNA and no formation of secondary structure in the oligonucleotide sequence by homology search. The evaluation of whether the designed antisense oligonucleotide is functional or not can be determined by introducing the antisense oligonucleotide into a suitable cell and measuring the amount of the target mRNA, for example by northern blotting or RT-PCR, or the amount of the target protein, for example by western blotting or fluorescent antibody technique, to confirm the effect of expression inhibition

Another method includes the triple helix technique. This technique involves forming a triple helix on the targeted intra-nuclear DNA sequence, thereby regulating its gene expression, mainly at the transcription stage. The oligonucleotide is designed mainly in the gene region involved in the transcription and inhibits the transcription and the production of the protein of the present invention. Such RNA, DNA and oligonucleotide can be produced using known synthesizers.

The oligonucleotide may be introduced into the cells containing the target nucleic acid sequence by any of DNA transfection methods such as calcium phosphate method, electroporation, lipofection, microinjection, or gene transfer methods including the use of gene transfer vectors such as viruses. An antisense oligonucleotide expression vector can be prepared using a suitable retrovirus vector, then the expression vector can be introduced into the cells containing the target nucleic acid sequence by contacting the vector with the cells in vivo or ex vivo.

The DNA of the present invention can be used in the antisense RNA/DNA technique or the triple helix technique to inhibit promotion of STAT6 activation mediated by the protein of the present invention.

The antisense oligonucleotide against the gene encoding the protein of the present invention is useful as a medicament to treat or prevent diseases characterized by undesirable activation of STAT6, such as allergic disease, inflammation, autoimmune diseases, diabetes, hyperlipidemia, infectious diseases (e.g., HIV infection) and cancers. Thus, the present invention also includes a pharmaceutical composition which comprises the above antisense oligonucleotide as an active ingredient. The antisense oligonucleotide can also be used to detect such diseases using northern hybridization or PCR.

The present invention also includes a ribozyme which inhibits STAT6 activation. A ribozyme is an RNA capable of recognizing a nucleotide sequence of a nucleic acid and cleaving the nucleic acid (see e.g., Hiroshi Yanagawa, “Jikken Igaku (Experimental Medicine) Bioscience 12: New Age of RNA). The ribozyme can be produced so that it cleaves the selected target RNA (e.g., mRNA encoding the protein of the present invention). Based on the nucleotide sequence of the DNA encoding the protein of the present invention, the ribozyme specifically cleaving the mRNA of the protein of the present invention can be designed. Such ribozyme has a complementary sequence to the mRNA for the protein of the present invention, complementarily associates with the mRNA and then cleaves the mRNA, which results in reduction or entire loss of the expression of the protein of the present invention. The level of the reduction of the expression is dependent on the level of the ribozyme expression in the target cells.

There are two types of ribozyme commonly used: a hammerhead ribozyme and a hairpin ribozyme. In particular, hammerhead ribozymes have been well studied regarding their primary and secondary structure necessary for their cleavage activity, and those skilled in the art can easily design the ribozymes nucleotides solely on the nucleotide sequence information for the DNA encoding the protein of the present invention [see e.g., Iida et al., Saibou Kougaku Vol.16, No.3, p.438-445 (1997); Ohkawa & Taira, Jikken Igaku (Experimental Medicine) Vol.12, No.12, p.83-88 (1994)]. It is known that the hammerhead ribozymes have a structure consisting of two recognition sites (recognition site I and recognition site II forming a chain complementary to target RNA) and an active site, and cleave the target RNA at the 3′end of its sequence NUX (wherein N is A or G or C or U, and X is A or C or U) after the formation of a complementary pair with the target RNA in the recognition sites. In particular, the sequence GUC (or GUA) has been found to have the highest activity [see e.g., Koizumi, M. et al., Nucl. Acids Res. 17:7059-7071 (1989); Iida et al., Saibou Kougaku Vol.16, No.3, p.438-445 (1997); Ohkawa & Taira, Jikken Igaku (Experimental Medicine) Vol.12, No.12, p.83-88 (1994); Kawasaki & Taira, Jikken Igaku (Experimental Medicine) Vol.18, No.3, p.381-386 (2000)].

Therefore the sequence GTC (or GTA) is searched out, and a ribozyme is designed to form several, up to 10 to 20 complementary base pairs around that sequence. The suitability of the designed ribozyme can be evaluated by checking whether the prepared ribozyme can cleave the target mRNA in vitro according to the method described for example in Ohkawa & Taira, Jikken Igaku (Experimental Medicine) Vol.12, No.12, p.83-88 (1994). The ribozyme can be prepared by methods known in the art to synthesize RNA molecules.

Alternatively, the sequence of the ribozyme can be synthesized on a DNA synthesizer and inserted into various vectors containing a suitable RNA polymerase promoter (e.g., T7 or SP6) to enzymatically synthesize an RNA molecule in vitro. Such ribozymes can be introduced into cells by gene transfer methods such as microinjection. Another method involves inserting a ribozyme DNA into a suitable expression vector and introducing the vector into cell strains, cells or tissues. Suitable vectors can be used to introduce the ribozyme into a selected cell. Examples of vectors commonly used for such purpose include plasmid vectors and animal virus vectors (e.g., retrovirus, adenovirus, herpes or vaccinia virus vectors). Such ribozymes are capable of inhibiting promotion of STAT6 activation mediated by the protein of the present invention.

DNA encoding the protein which acts to promote STAT6 activation of the present invention was obtained by a method which comprises using the oligo-capping method to construct a full-length cDNA library, and using a signal factor indicative of the presence of a protein having the function. An example of such a signal factor is a reporter gene.

Methods using a cDNA library containing a lot of non-full-length cDNAs are inefficient in obtaining many genes (cDNAs) having functions. Therefore libraries with a high ratio of the number of the full-length cDNA clones to the total number of the clones are necessary. “Full-length cDNA” refers to a complete DNA copy of mRNA from a gene. The cDNA libraries produced using the oligo-capping method contain full-length cDNA clones in a ratio of 50 to 80%, namely, a 5 to 10-fold increase in full-length cDNA clones compared to the cDNA libraries produced by prior art methods (Sumio Sugano, the monthly magazine BIO INDUSTRY Vol.16, No.11, p.19-26). Full-length cDNA clones are essential for protein expression in functional analyses of genes, and full-length cDNA clones themselves are very important materials for activity measurement. Thus, cloning of full-length cDNA is necessary for functional analyses of genes. Sequencing of the cDNA not only provides important information for establishing the primary sequence of the protein encoded by the cDNA, but also reveals the entire exon sequence. Thus, the full-length cDNA provides valuable information for identifying a gene, such as information for determining the primary sequence of a protein, exon-intron structure, the transcription initiation site of mRNA, the location of a promoter, etc.

The construction of full-length cDNA libraries by the oligo-capping method can be carried out, for example, according to the method described in “Shin Idenshi Kougaku Handbook (New Genetic Engineering Handbook)”, the third edition (1999), an extra issue of “Jikken Igaku (Experimental Medicine)”, YODOSHA CO., LTD. The reporter gene indicative of the presence of a protein having a function contains one or more suitable expression regulation sequence portion to which a protein factor such as a transcriptional factor can bind, and a structural gene portion which allows the measurement of the activation of the proteins factor The structural gene portion may encode any peptide or protein so long as those skilled in the art can measure the activity or amount of its expression product (including the amount of the mRNA produced). For example, chloramphenicol acetyltransferase, β-galactosidase, luciferase, etc., can be used and their enzymatic activity measured.

The oligo-capping method involves substituting a cap structure with a synthetic oligo sequence by using BAP, TAP and an RNA ligase, as described in Suzuki & Sugano, “Shin Idenshi Kougaku Handbook (New Genetic Engineering Handbook)”, the third edition (1999), an extra issue of “Jikken Igaku (Experimental Medicine)”, YODOSHA CO., LTD.

To obtain DNA encoding the protein which promotes STAT6 activation of the present invention, either an in vitro system or a cell-based system, preferably a cell-based system, is used. Examples of such cells include cells of prokaryotes such as E. coli, microorganisms such as yeast and fungi, as well as insects and animals. Preferred examples include animal cells, in particular, 293-EBNA cells and NIH3T3 cells.

Examples of reporter genes indicative of the presence of a protein having a function include reporter genes containing a CREB (cAMP responsive element binding protein) binding sequence or AP-1 (activator protein-1) binding sequence at the expression regulation sequence region of the reporter genes, in addition to the STAT6 reporter genes described herein. For example, if a gene capable of activating CREB is to be obtained, a CREB-dependent reporter plasmid and a full-length cDNA clone produced by the oligo-capping method can be cotransfected into cells, and a plasmid having increased reporter activity can be selected from the cells to attain the purpose. If a gene capable of inhibiting CREB is to be obtained, a CREB-dependent reporter plasmid and a full-length cDNA clone produced by the oligo-capping method can be cotransfected into cells, and a plasmid having decreased reporter activity can be selected from the cells to attain the purpose. These procedures may be carried out in the presence of a certain stimulus to the cells. The cDNA to be transfected into the cells may be a single clone or multiple clones which may be transfected simultaneously. Alternatively, a screening system for obtaining a gene capable of inhibiting STAT6 activation can also be constructed by cotransfecting a full-length cDNA and a reporter gene into cells) stimulating the cells by IL-4, IL-13 or the like, and selecting a clone having subnormally increased reporter activity.

Further, because the cDNA of the present invention is full-length, its 5′ end sequence is the transcription initiation site of the corresponding mRNA. Therefore the cDNA sequence can be used to identify the promoter region of the gene by comparing the cDNA with the genomic nucleotide sequence. Genomic nucleotide sequences are available from various databases when the sequences have been deposited in the databases. Alternatively, the cDNA can also be used to clone the desired sequence from a genomic library, for example, by hybridization, and determine its nucleotide sequence. Thus, by comparing the nucleotide sequence of the cDNA of the present invention with a genomic sequence, the promoter region of the gene located upstream the cDNA can be identified. In addition, the promoter fragment thus identified can be used to construct a reporter plasmid for evaluating the expression of the gene. In general, the DNA fragment spanning 2 kb (preferably 1 kb) upstream from the transcription initiation site can be inserted upstream of the reporter gene to produce the reporter plasmid. The reporter plasmid can be used to screen for a compound which enhances or reduces the expression of the gene. For example, such screening can be carried out by transforming a suitable cell with the reporter plasmid, culturing the transformed cell for a certain period of time, adding a certain amount of a test compound, measuring the reporter activity expressed by the cell after a certain period of time, and comparing the activity with that of a cell to which the test compound has not been added. These methods are also included in the scope of the present invention.

The present invention also relates to a computer-readable medium on which a sequence data set has been stored, said sequence data set comprising at least one nucleotide sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481 and 483 and/or at least one amino acid sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482 and 484.

In another aspect, the present invention relates to a method for calculating a homology, which comprises comparing data on the above medium with data of other nucleotide sequences. Thus, the polynucleotide and amino acid sequence of the present invention provide valuable information for determining their secondary and tertiary structure, e.g., information for identifying other sequence having a similar function and high homology. These sequences are stored on the computer-readable medium, then a database is searched using data stored in a known macromolecule structure program and a known search tool such as GCG program package (Devereux, J. et al, Nucleic Acids Research 12(1):387 (1984)). In this manner, a sequence in a database having a certain homology can be easily found.

The computer-readable medium may be any composition of materials used to store information or data. Examples of such media include commercially available floppy disks, tapes, chips, hard disk, compact disks and video disks. The data on the medium allows a method for calculating a homology by comparing the data with other nucleotide sequence data. This method comprises the steps of providing a first polynucleotide sequence containing the polynucleotide sequence of the present invention for the computer-readable medium, and then comparing the first polynucleotide sequence with at least one-second polynucleotide or polypeptide sequence to identify the homology.

The present invention also relates to an insoluble substrate to which polynucleotide comprising all or part of the nucleotide sequences selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481 and 483 are fixed. A plurality of the various polynucleotides which are DNA probes are fixed on a specifically processed solid substrates such as slide glass to form a DNA microarray and then a labeled target polynucleotide is hybridized with the fixed polynucleotides to detect a signal from each of the probes. The data obtained is analyzed and the gene expression is determined.

The present invention further relates to an insoluble substrate to which polypeptides comprising all or part of the amino acid sequences of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482 and 484 are fixed. By mixing organism-derived cell extract with the insoluble substrate on which these proteins are fixed, it is possible to isolate or identify substances captured on the insoluble substrate that can be expected to be useful in diagnosis or drug development.

### EXAMPLES

The following examples further illustrate, but do not limit the present invention.

### Example 1

Construction of a Full-length cDNA Library Using the Oligo-capping Method

(1) Preparation of RNA from Human Lung Fibroblasts (Cryo NHLF)

Human lung fibroblasts (Cryo NHLF: purchased from Sanko Junyaku Co., Ltd.) were cultured according to the attached protocol. After repeating subculturing the cells to obtain fifty 10 cm dishes containing the resulting culture, the cells were recovered with a cell scraper. Then, total RNA was obtained from the recovered cells by using the RNA extraction reagent ISOGEN (purchased from NIPPON GENE) according to the manufacture's protocol. Then, poly A⁺ RNA was obtained from the total RNA by using an oligo-dT cellulose column according to Maniatis et al., supra.

(2) Construction of a Full-length cDNA Library by the Oligo-capping Method

A full-length cDNA library was constructed from the above poly A⁺ RNA by the oligo-capping method according to the method of Sugano S. et al. [e.g., Maruyama, K. & Sugano, S., Gene, 138:171-174 (1994); Suzuki, Y et al., Gene, 200:149-156 (1997); Suzuki, Y. & Sugano, S. “Shin Idenshi Kougaku Handbook (New Genetic Engineering Handbook)”, the third edition (1999), an extra issue of “Jikken Igaku (Experimental Medicine)”, YODOSHA CO., LTD.].

(3) Preparation of Plasmid DNA

The full-length cDNA library constructed as above was transfected into E. coli strain TOP 10 by electroporation, then spread on LB agar medium, and incubated overnight at 37° C. Then, using QIAwell 96 Ultra Plasmid Kit (QIAGEN) according to the manufacturer's protocol, the plasmids were recovered from the colonies grown on ampicillin-containing LB agar medium.

### Example 2

Cloning of DNA Capable of Promoting STAT6 Activation

(1) Screening of the cDNA Encoding the Protein Capable of Promoting STAT6 Activation

NIH3T3 cells (purchased from Dainippon Pharmaceutical) were grown to 1×10⁴ cells/well in a 96 well plate for cell culture for 24 hours at 37° C. (in the presence of 5% CO₂) using 10% FBS containing IMDM medium. Then, 100 ng of luciferase reporter plasmid N4×8-luc having a STAT6 response sequence and 2 μl of the full-length cDNA prepared in above Example 1.(3) were cotransfected into the cells in a well using FuGENE 6 (purchased from Roche) according to the manufacturer's protocol. The luciferase reporter plasmid N4×8-luc having the STAT6 response sequence was constructed as follows. With reference to the oligonucleotide sequence to which an activated STAT6 binds specifically, found by Ohmori et al. [J. Immunol. 157, 2058-2065 (1996)], oligonucleotides having the following sequences were synthesized:

(SEQ ID NO:485)
5′-TCGAGCTCTTCTTCCCAGGAACTCAATG-3′,
(SEQ ID NO:486)

5′-TCGACATTGAGTTCCTGGGAAGAAGAGC-3′

The synthesized oligonucleotides were dissolved in sterile water to be 1μg/μl, respectively, mixed in 10 μl lots, and adjusted the volume to 32 μl with sterile water. The solution was heated for 5 min at 90° C., and gradually cooled down to room temperature to prepare a double-stranded oligonucleotide solution. The solution was reacted with T4 polynucleotide kinase (Takara Shuzo) according to the attached manual, then the reaction product was purified in a usual manner. Separately, SV40 promoter region of pGL3-Promoter vector (Promega) was replaced by the HSV thymidine kinase promoter sequence (from −50 to +10) with Hind III site and BglII site to construct a vector tk-luc. The aforesaid double-stranded oligonucleotide fragments were inserted into the XhoI site of this tk-luc vector using T4 DNA ligase (GIBCO/BRL). The obtained clones were sequenced according to a usual method, and clones in which plural oligonucleotide fragments were inserted were selected. A clone with at most 4 inserted fragments was obtained, which was named as N4×4-luc. The four-interlinked DNA fragments were excised from the N4×4-luc with a XhoI and a BglII site and purified to be inserted into a BamHI and a XhoI site of pBluescript II KS+ (Stratagene). The four-interlinked DNA fragments were excised from this plasmid with KpnI and SpeI and inserted into a KpnI and a NheI site of N4×4-luc plasmid to finally obtain N4×8-luc.

After transfection, the cells were cultured for 48 hours at 37° C., followed by 6 hours of culture with addition of mouse IL-4 (Immuno-Biological Laboratories) to a final concentration of 0.5 ng/ml. The reporter activity of STAT6 (luciferase activity) was measured using long-term luciferase assay system, PIKKA GENE LT2.0 (TOYO INK) according to the attached manufacturer's instructions. The luciferase activity was measured using Wallac ARVO™ST 1420 MULTILABEL COUNTER (Perkin Elmer).

(2) DNA Sequencing

The above screening was carried out for 115, 000 clones, and plasmids showing a 3-fold or more increase in luciferase activity compared to that of the control experiment (luciferase activity of the cell into which vacant vector pME18S-FL3 is introduced instead of full-length cDNA) were selected. One pass sequencing was carried out from the 5′ end of the cloned cDNA (sequencing primer: 5′-CTTCTGCTCTAAAAGCTGCG-3′ (SEQ ID NO: 487)) and from the 3′ end (sequencing primer: 5′-CGACCTGCAGCTCGAGCACA-3′ (SEQ ID NO: 488)) so that as long sequence as possible is determined. The sequencing was carried out using the reagent Thermo Sequenase II Dye Terminator Cycle Sequencing Kit (Amersham Pharmacia Biotech) or BigDye Terminator Cycle Sequencing FS Ready Reaction Kit (Applied Biosystems) and the device ABI PRISM 377 sequencer or ABI PRISM 3100 sequencer according to the manufacturer's instructions.

(3) Database Analysis of the Obtained Clones

BLAST (Basic local alignment search tool) searching [S. F. Altschul et al., J. Mol. Biol., 215:403-410 (1990)] was carried out in GenBank for the obtained nucleotide sequence. The results showed that 242 clones represented 112 genes encoding new proteins capable of promoting STAT6 activity.

(4) Full-length Sequencing

The full-length DNA sequences for the 242 new clones were determined (SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 451, 453, 455, 457, 459, 461, 463, 465, 467, 469, 471, 473, 475, 477, 479, 481 and 483). The amino acid sequences of the protein coding regions (open reading frames) were deduced (SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482 and 484).

Regarding nucleotide sequences obtained by the above screening, data concerning the luciferase activity of each nucleotide is indicated below. Values for activity are indicated as a ratio of the luciferase activity of cells into which a nucleotide according to each SEQ ID NO. are introduced, divided by luciferase activity of cells into which pME18S-FL3 is introduced. Luciferase activity can be measured in respect of nucleotides according to SEQ ID NOs. not shown in the table, by a similar method.

| SEQ ID NO. (DNA) | Activity *) | SEQ ID NO. (DNA) | Activity *) |
|---|---|---|---|
| 4 | 7.1 | 181 | 4.7 |
| 6 | 127.5 | 183 | 92.3 |
| 8 | 4.2 | 185 | 43.1 |
| 10 | 10.0 | 189 | 3.4 |
| 12 | 9.7 | 193 | 92.5 |
| 14 | 3.5 | 203 | 5.6 |
| 18 | 183.1 | 205 | 3.5 |
| 20 | 50.1 | 207 | 3.0 |
| 26 | 33.5 | 209 | 9.6 |
| 30 | 20.2 | 211 | 5.3 |
| 32 | 5.0 | 217 | 1521.5 |
| 34 | 21.1 | 219 | 12.7 |
| 36 | 21.1 | 223 | 580.4 |
| 38 | 3.1 | 231 | 4.0 |
| 42 | 7.2 | 237 | 105.1 |
| 50 | 18.5 | 241 | 54.3 |
| 56 | 4.7 | 261 | 14.7 |
| 58 | 3.8 | 263 | 12.5 |
| 62 | 20.3 | 273 | 16.6 |
| 64 | 3.2 | 275 | 17.0 |
| 66 | 15.1 | 279 | 28.5 |
| 69 | 11.7 | 281 | 3.9 |
| 71 | 7.9 | 283 | 16.7 |
| 73 | 5.6 | 291 | 13.0 |
| 75 | 3.1 | 293 | 25.8 |
| 77 | 7.1 | 295 | 31.0 |
| 79 | 15.3 | 303 | 22.5 |
| 85 | 11.5 | 317 | 5.1 |
| 87 | 13.1 | 323 | 12.3 |
| 89 | 10.2 | 325 | 6.1 |
| 93 | 5.7 | 327 | 5.3 |
| 95 | 8.9 | 343 | 26.7 |
| 97 | 13.1 | 345 | 3.7 |
| 101 | 8.0 | 349 | 24.3 |
| 105 | 6.3 | 353 | 155.4 |
| 107 | 3.1 | 355 | 66.1 |
| 109 | 7.1 | 357 | 15.6 |
| 111 | 3.7 | 371 | 12.3 |
| 113 | 4.3 | 373 | 10.2 |
| 115 | 8.6 | 375 | 8.9 |
| 117 | 10.1 | 377 | 38.6 |
| 121 | 4.5 | 383 | 34.1 |
| 125 | 9.2 | 397 | 32.6 |
| 127 | 5.0 | 399 | 10.5 |
| 129 | 14.8 | 413 | 3.4 |
| 131 | 13.5 | 415 | 18.4 |
| 133 | 12.7 | 417 | 13.4 |
| 137 | 20.3 | 423 | 4.8 |
| 139 | 4.1 | 425 | 11.4 |
| 141 | 11.7 | 429 | 3.2 |
| 149 | 9.9 | 431 | 3.8 |
| 151 | 104.7 | 433 | 8.8 |
| 159 | 19.1 | 435 | 5.1 |
| 163 | 3.5 | 441 | 4.5 |
| 165 | 3.5 | 455 | 3.3 |
| 167 | 3.0 | 465 | 13.3 |
| 171 | 4.8 | 469 | 6.5 |
| 175 | 17.7 | 471 | 147.7 |
| 177 | 22.6 | 481 | 6.4 |

Activity = (luciferase activity of the gene of each SEQ ID NO:)/(luciferase activity of pME18S-FL3)

### Example 3

Screening Compounds Inhibiting Promotion of STAT6 Activity

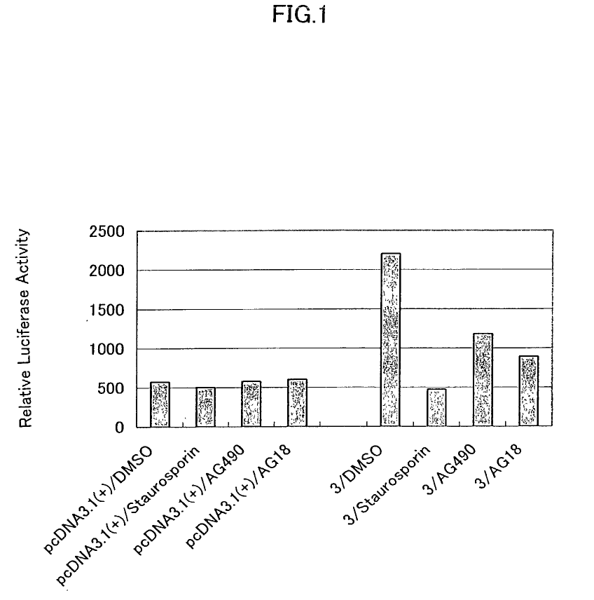

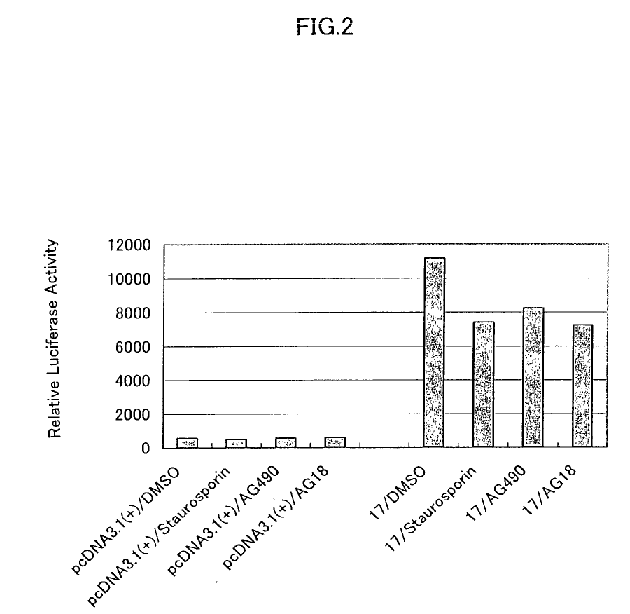

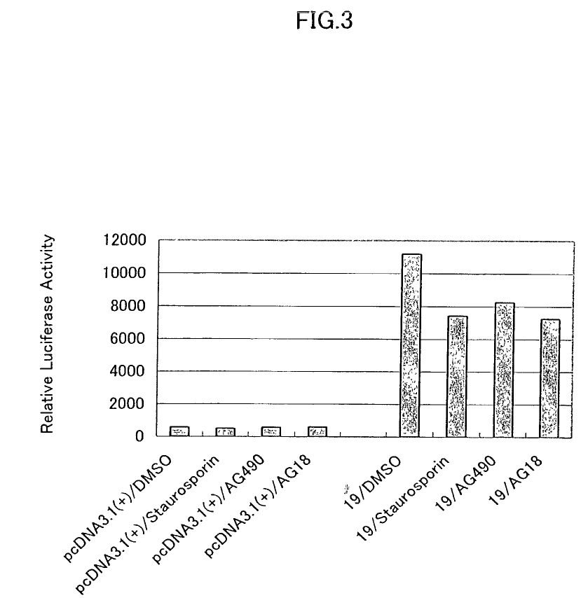

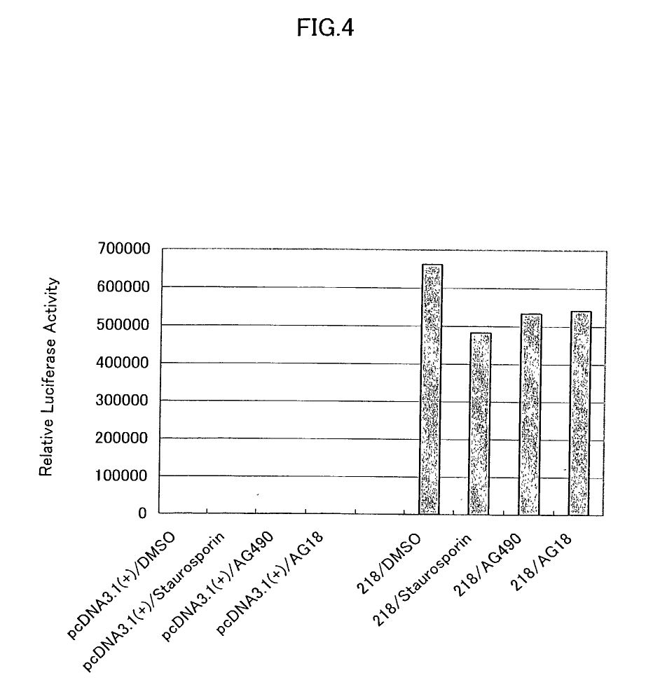

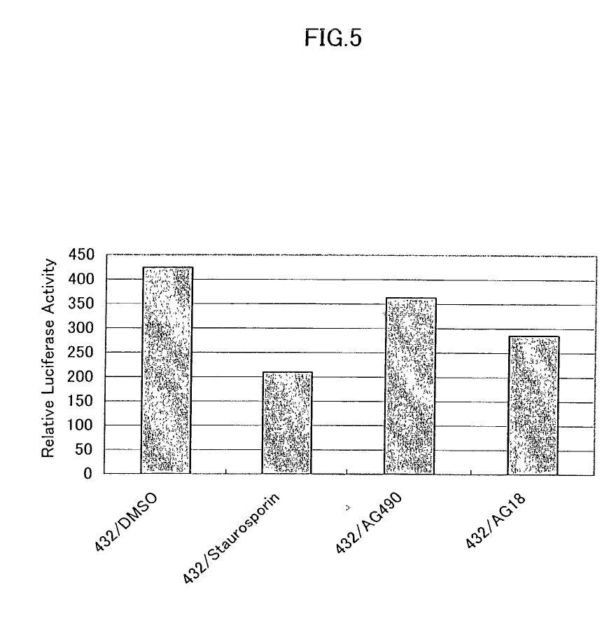

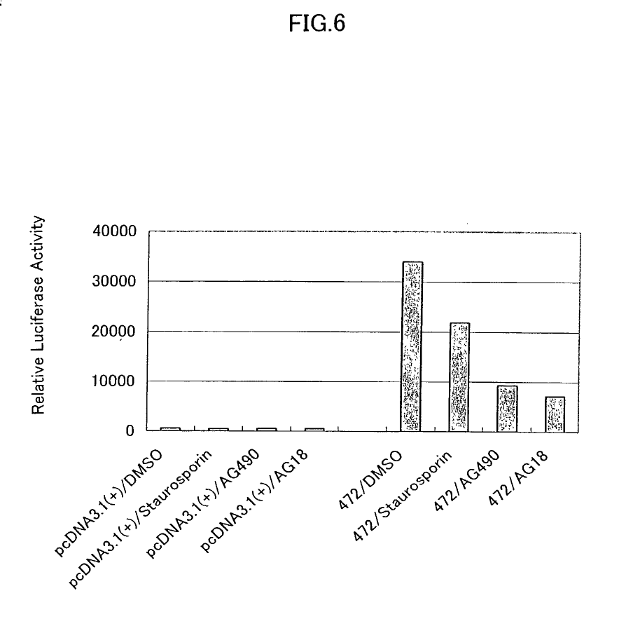

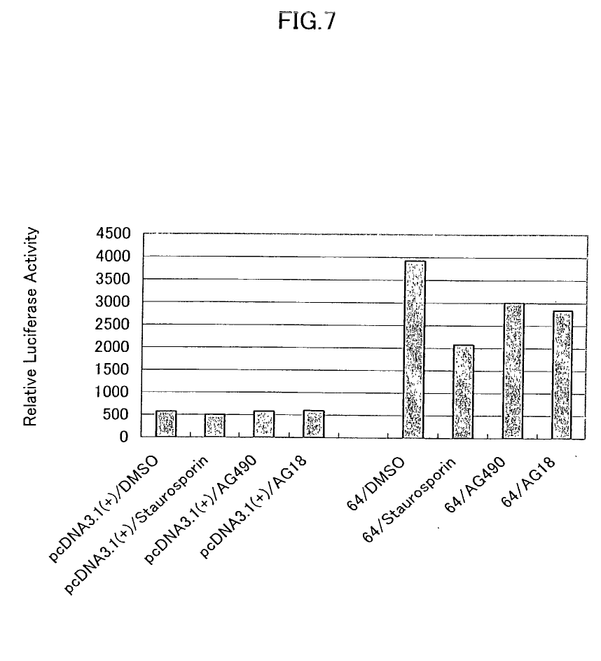

NIH3T3 cells were seeded on 10% FBS containing IMDM medium in a 96-well cell culture plate to a final cell density of 1×10⁴ cells/100 μl/well, and cultured for 24 hours at 37° C. in the presence of 5% CO₂. Then, 30 ng of the plasmid containing the nucleotide encoding the STAT6 activation-promoting protein of SEQ ID NO: 3, 17, 19, 218, 432 or 472, or the nucleotide of SEQ ID NO: 64, and 100 ng of the luciferase reporter plasmid having the STAT6 response sequence were cotransfected into the cells in a well using FuGENE 6. After 48 hours, AG18, AG490, or staurosporin (purchased form CALBIOCHEM) known to be a protein kinase inhibitor was added to the culture to a final concentration of 20 μM, 20 μM, 30 μM, respectively. After 30 min of culture at 37° C., followed by 6 hours of culture with addition of mouse IL-4 to a final concentration of 1 ng/ml, the reporter activity was measured using PIKKA GENE LT2.0. The results showed that the AG18, AG490, and staurosporin inhibited the expression of the reporter gene in the well in which the plasmid containing a nucleotide encoding the STAT6 activation-promoting protein of SEQ ID NO: 3, 17, 19, 218, 432 or 472, or nucleotide according to SEQ ID NO: 64 was introduced while no expression was inhibited at all in the well in which a control plasmid pcDNA3.1 (+) (Invitrogen) was introduced (FIGS. 1 to 7).

Similarly, control of expression of a reporter gene can be confirmed in respect of genes coding for other amino acid sequences by a similar method.

### INDUSTRIAL APPLICABILITY

As described above, the present invention provides industrially highly useful proteins capable of promoting STAT6 activity and genes encoding the proteins. The proteins of the present invention and the genes encoding the proteins allow not only screening for compounds useful for treating and preventing diseases associated with the excessive activation or inhibition of STAT6, but also production of diagnostics for such diseases. The genes of the present invention are also useful as a gene source used for gene therapy.

All publications, patents and patent applications cited herein are incorporated herein in their entirety.

1. A purified protein selected from the group comprising of:
(a) a protein which consists of an amino acid sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482 and 484; and
(b) a protein that promotes STAT6 activation and consists of an amino acid sequence having at least one amino acid deletion, substitution or addition in an amino acid sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, and 484.

2. A purified protein that promotes STAT6 activation and comprises an amino acid sequence having at least 95% identity to the protein according to claim 1 over the entire length thereof.

3. An isolated polynucleotide which comprises a nucleotide sequence encoding a protein selected from the group consisting of:
(a) a protein which comprises an amino acid sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482 and 484; and
(b) a protein that promotes STAT6 activation and consists of an amino acid sequence having at least one amino acid deletion, substitution or addition in an amino acid sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482 and 484.

4. An isolated polynucleotide comprising a polynucleotide sequence selected from the group consisting of:
(a) a polynucleotide sequence represented by any one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481 and 483; and a polynucleotide sequence complementary to said polynucleotide;
(b) a polynucleotide sequence encoding a protein that promotes STAT6 activation and hybridizing with a polynucleotide having any one of the polynucleotide sequences of (a) under stringent conditions; and
(c) a polynucleotide sequence which encodes a protein that promotes STAT6 activation, and which consists of a polynucleotide sequence having at least one nucleotide deletion, substitution or addition in a polynucleotide sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481 and 483.

5. An isolated polynucleotide comprising a nucleotide sequence which encodes a protein that promotes STAT6 activation and has at least 95% identity to any one of the polynucleotide sequences according to claim 3 over the entire length thereof.

6. An isolated polynucleotide comprising a nucleotide sequence which encodes a protein that promotes STAT6 activation and has at least 95% identity to any one of the polynucleotide sequences according to claim 4 over the entire length thereof.

7. A purified protein encoded by the polynucleotide according to any one of claims 3 to 6.

8. A recombinant vector which comprises a polynucleotide according to any one of claims 3 to 6.

9. A transformed cell which comprises the recombinant vector according to claim 8.

10. A membrane of the cell according to claim 9, when the protein according to claim 1 or 2 is a membrane protein.

11. A process for producing a protein comprising,
(a) culturing a transformed cell comprising any one of the isolated polynucleotides according to any one of claims 3 to 6 under conditions providing expression of the encoded protein; and
(b) recovering the protein from the culture.

12. A process for diagnosing a disease or susceptibility to a disease in a subject related to expression or activity of the protein of claim 1, 2 or 7 in a subject comprising:
(a) determining the presence or absence of a mutation in the nucleotide sequence encoding said protein in the genome of said subject; and/or
(b) analyzing the amount of expression of said protein in a sample derived from said subject, wherein a diagnosis of disease is made according to an increase or decrease in the amount of the protein expressed.

13. A method for screening compounds for activity as inhibitors or activators of STAT6, which comprises the steps of:
(a) providing a cell with a gene encoding a protein that promotes STAT6 activation, and a component that provides a detectable signal associated with activation of STAT6;
(b) culturing a transformed cell under conditions, which permit the expression of the gene in the transformed cell;
(c) contacting the transformed cell with one or more compounds; and
(d) measuring the detectable signal; and
(e) isolating or identifying as an activator compound and/or an inhibitor compound according to said detectable signal.

14. A process for producing a pharmaceutical composition, which comprises the steps of:
(a) providing a cell with a gene encoding a protein that promotes STAT6 activation, and a component capable of providing a detectable signal;
(b) culturing the transformed cell under conditions, which permit the expression of the gene in the transformed cell;
(c) contacting the transformed cell with one or more compounds;
(d) measuring the detectable signal;
(e) isolating or identifying as an activator compound and/or an inhibitor compound according to said detectable signal; and
(f) optimizing the isolated or identified compound as a pharmaceutical composition.

15. A kit for screening a compound for activity as an inhibitor or activator of STAT6, which comprises:
(a) a cell comprising a gene encoding a protein that promotes STAT6 activation, and a component that provides a detectable signal upon activation of STAT6; and
(b) reagents for measuring the detectable signal.

16. A monoclonal or polyclonal antibody that reacts with the protein according to claim 1, 2 or 7.

17. A process for producing a monoclonal or polyclonal antibody that reacts with the protein of claim 1, 2 or 7, which comprises administering the protein according to claim 1, 2 or 7, or epitope-bearing fragments thereof to a non-human animal.

18. An antisense oligonucleotide complementary to the polynucleotide according to any one of claims 3 to 6, which prevents expression of a protein that promotes STAT6 activation.

19. A ribozyme which inhibits STAT6 activation by cleavage of RNA that encodes the protein of claim 1, 2 or 7, or by cleavage of RNA that encodes some protein of the pathway that leads to STAT6 activation.

20. A method for treating a disease, which comprises administering to a subject an amount of a compound screened by the process according to claim 13, and/or a monoclonal or polyclonal antibody according to clam 16, and/or an antisense oligonucleotide according to claim 18 and/or a ribozyme according to claim 19 effective to treat a disease selected from the group consisting of allergic disease, inflammation, autoimmune diseases, diabetes, hyperlipidemia, infectious disease and cancers.

21. A pharmaceutical composition produced according to claim 14 as inhibiting or activating STAT6 activation.

22. A pharmaceutical composition according to claim 21 for the treatment of allergic disease, inflammation, autoimmune diseases, diabetes, hyperlipidemia, infectious disease and cancers.

23. A method of treating allergic disease, inflammation, autoimmune diseases, diabetes, hyperlipidemia, infectious disease and cancers, which comprising administering a pharmaceutical composition produced according to claim 14 to a patient suffering from a disease related to STAT6 activation.

24. A pharmaceutical composition according to claim 21 for the treatment of Th1 hyperactive diseases.

25. A method of treating Th1 hyperactive diseases, which comprises administering a pharmaceutical composition produced according to claim 14 to a patient suffering from a disease related to inhibition of STAT6 activation.

26. A pharmaceutical composition which comprises a monoclonal or polyclonal antibody according to claim 16 as an active ingredient.

27. A pharmaceutical composition which comprises an antisense oligonucleotide according to claim 18 as an active ingredient.

28. The pharmaceutical composition according to claim 26 or 27, wherein the target disease is selected from the group consisting of allergic disease, inflammation, autoimmune diseases, diabetes, hyperlipidemia, infectious diseases and cancers.

29. A computer-readable medium on which a sequence data set has been stored, said sequence data set comprising at least one nucleotide sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481 and 483 and/or at least one amino acid sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482 and 484.

30. A method for calculating identity to other nucleotide sequences and/or amino acid sequences, which comprises comparing data on a medium according to claim 29 with data of said other nucleotide sequences and/or amino acid sequences.

31. An insoluble substrate to which polynucleotide comprising all or part of the nucleotide sequences selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481 and 483 are fixed.

32. An insoluble substrate to which polypeptides comprising all or a part of the amino acid sequences selected from the group consisting of SEQ ID NOS: 1, 3, 5, 7, 9, 11, 13, 15, 17, 19, 21 23, 25, 27, 29, 31, 33, 35 37 39 41 43 45, 47, 49, 51, 53, 55, 57, 59, 61, 63, 65, 67, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482 and 484 are fixed.
